(19) United States
(12) Patent Application Publication
(10) Pub. No.: US 2003/0215807 A1
(43) Pub. Date: Nov. 20, 2003

(54) Method and system for normalization of micro array data based on local normalization of rank-ordered, globally normalized data

(76) Inventors: Paul K. Wolber, Los Altos, CA (US); Karen W. Shannon, Los Gatos, CA (US); Stephanie B. Fulmer-Smentek, Sunnyvale, CA (US); Charles D. Troup, Pleasanton, CA (US); Douglas A. Amorese, Los Altos, CA (US); Nicholas M. Sampas, San Jose, CA (US); Srinka Ghosh, San Francisco, CA (US); Scott D. Connell, Saratoga, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/143,547

(22) Filed: May 9, 2002

(51) Int. Cl. C12Q 1/68; G06F 19/00; G01N 33/48; G01N 33/50

(52) U.S. Cl. 435/6; 702/19; 702/20

(57) ABSTRACT

A method and system for normalizing two or more molecular array data sets. Input molecular array data sets are separately globally normalized by, for example, dividing the feature-signal magnitudes of each data set by the geometric mean of the feature-signal magnitudes of the data set. The globally normalized feature signal magnitudes within each data set are ranked in ascending order. A numeric function is created that relates feature-signal magnitudes of the data sets. Only a subset of the features, obtained by selecting features that are similarly ranked in the separate feature-signal-magnitude rankings for the data sets, is used to construct the numeric function. The numeric function is smoothed by one of many possible different smoothing procedures. The smoothed numeric function is used to rescale the feature-signal magnitude in one data set to the feature-signal magnitude of another data set, or to normalize the data sets to one another by distributing correction terms amongst the feature-signal magnitudes for a feature in each data set.

### TECHNICAL FIELD

The present invention relates to the analysis of data obtained by scanning molecular arrays and, in particular, to a method and system for normalizing two or more sets of data obtained by scanning a molecular array at two or more optical wavelengths or radioactive emission energies, as well as for normalizing data sets obtained from different molecular arrays.

### BACKGROUND OF THE INVENTION

The present invention is related to processing of data scanned from arrays. Array technologies have gained prominence in biological research and are likely to become important and widely used diagnostic tools in the healthcare industry. Currently, molecular-array techniques are most often used to determine the concentrations of particular nucleic-acid polymers in complex sample solutions. Molecular-array-based analytical techniques are not, however, restricted to analysis of nucleic acid solutions, but may be employed to analyze complex solutions of any type of molecule that can be optically or radiometrically scanned and that can bind with high specificity to complementary molecules synthesized within, or bound to, discrete features on the surface of an array. Because arrays are widely used for analysis of nucleic acid samples, the following background information on arrays is introduced in the context of analysis of nucleic acid solutions following a brief background of nucleic acid chemistry.

Deoxyribonucleic acid (“DNA”) and ribonucleic acid (“RNA”) are linear polymers, each synthesized from four different types of subunit molecules. The subunit molecules for DNA include: (1) deoxy-adenosine, abbreviated “A,” a purine nucleoside; (2) deoxy-thymidine, abbreviated “T,” a pyrimidine nucleoside; (3) deoxy-cytosine, abbreviated “C,” a pyrimidine nucleoside; and (4) deoxy-guanosine, abbreviated “G,” a purine nucleoside. The subunit molecules for RNA include: (1) adenosine, abbreviated “A,” a purine nucleoside; (2) uracil, abbreviated “U,” a pyrimidine nucleoside; (3) cytosine, abbreviated “C,” a pyrimidine nucleoside; and (4) guanosine, abbreviated “G,” a purine nucleoside. FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108. When phosphorylated, subunits of DNA and RNA molecules are called “nucleotides” and are linked together through phosphodiester bonds 110-115 to form DNA and RNA polymers. A linear DNA molecule, such as the oligomer shown in FIG. 1, has a 5′ end 118 and a 3′ end 120. A DNA polymer can be chemically characterized by writing, in sequence from the 5′ end to the 3′ end, the single letter abbreviations for the nucleotide subunits that together compose the DNA polymer. For example, the oligomer 100 shown in FIG. 1 can be chemically represented as “ATCG.” A DNA nucleotide comprises a purine or pyrimidine base (e.g. adenine 122 of the deoxy-adenylate nucleotide 102), a deoxy-ribose sugar (e.g. deoxy-ribose 124 of the deoxy-adenylate nucleotide 102), and a phosphate group (e.g. phosphate 126) that links one nucleotide to another nucleotide in the DNA polymer. In RNA polymers, the nucleotides contain ribose sugars rather than deoxy-ribose sugars. In ribose, a hydroxyl group takes the place of the 2′ hydrogen 128 in a DNA nucleotide. RNA polymers contain uridine nucleosides rather than the deoxy-thymidine nucleosides contained in DNA. The pyrimidine base uracil lacks a methyl group (130 in FIG. 1) contained in the pyrimidine base thymine of deoxy-thymidine.

The DNA polymers that contain the organization information for living organisms occur in the nuclei of cells in pairs, forming double-stranded DNA helixes. One polymer of the pair is laid out in a 5′ to 3′ direction, and the other polymer of the pair is laid out in a 3′ to 5′ direction. The two DNA polymers in a double-stranded DNA helix are therefore described as being anti-parallel. The two DNA polymers, or strands, within a double-stranded DNA helix are bound to each other through attractive forces including hydrophobic interactions between stacked purine and pyrimidine bases and hydrogen bonding between purine and pyrimidine bases, the attractive forces emphasized by conformational constraints of DNA polymers. Because of a number of chemical and topographic constraints, double-stranded DNA helices are most stable when deoxy-adenylate subunits of one strand hydrogen bond to deoxy-thymidylate subunits of the other strand, and deoxy-guanylate subunits of one strand hydrogen bond to corresponding deoxy-cytidilate subunits of the other strand.

FIGS. 2A-B illustrate the hydrogen bonding between the purine and pyrimidine bases of two anti-parallel DNA strands. FIG. 2A shows hydrogen bonding between adenine and thymine bases of corresponding adenosine and thymidine subunits, and FIG. 2B shows hydrogen bonding between guanine and cytosine bases of corresponding guanosine and cytosine subunits. Note that there are two hydrogen bonds 202 and 203 in the adenine/thymine base pair, and three hydrogen bonds 204-206 in the guanosine/cytosine base pair, as a result of which GC base pairs contribute greater thermodynamic stability to DNA duplexes than AT base pairs. AT and GC base pairs, illustrated in FIGS. 2A-B, are known as Watson-Crick (“WC”) base pairs.

Two DNA strands linked together by hydrogen bonds forms the familiar helix structure of a double-stranded DNA helix. FIG. 3 illustrates a short section of a DNA double helix 300 comprising a first strand 302 and a second, anti-parallel strand 304. The ribbon-like strands in FIG. 3 represent the deoxyribose and phosphate backbones of the two anti-parallel strands, with hydrogen-bonding purine and pyrimidine base pairs, such as base pair 306, interconnecting the two strands. Deoxy-guanylate subunits of one strand are generally paired with deoxy-cytidilate subunits from the other strand, and deoxy-thymidilate subunits in one strand are generally paired with deoxy-adenylate subunits from the other strand. However, non-WC base pairings may occur within double-stranded DNA.

Double-stranded DNA may be denatured, or converted into single stranded DNA, by changing the ionic strength of the solution containing the double-stranded DNA or by raising the temperature of the solution. Single-stranded DNA polymers may be renatured, or converted back into DNA duplexes, by reversing the denaturing conditions, for example by lowering the temperature of the solution containing complementary single-stranded DNA polymers. During renaturing or hybridization, complementary bases of anti-parallel DNA strands form WC base pairs in a cooperative fashion, leading to reannealing of the DNA duplex. Strictly A-T and G-C complementarity between anti-parallel polymers leads to the greatest thermodynamic stability, but partial complementarity including non-WC base pairing may also occur to produce relatively stable associations between partially-complementary polymers. In general, the longer the regions of consecutive WC base pairing between two nucleic acid polymers, the greater the stability of hybridization between the two polymers under renaturing conditions.

The ability to denature and renature double-stranded DNA has led to the development of many extremely powerful and discriminating assay technologies for identifying the presence of DNA and RNA polymers having particular base sequences or containing particular base subsequences within complex mixtures of different nucleic acid polymers, other biopolymers, and inorganic and organic chemical compounds. One such methodology is the array-based hybridization assay. FIGS. 4-7 illustrate the principle of the array-based hybridization assay. An array (402 in FIG. 4) comprises a substrate upon which a regular pattern of features is prepared by various manufacturing processes. The array 402 in FIG. 4, and in subsequent FIGS. 5-7, has a grid-like 2-dimensional pattern of square features, such as feature 404 shown in the upper left-hand corner of the array. Each feature of the array contains a large number of identical oligonucleotides covalently bound to the surface of the feature. These bound oligonucleotides are known as probes. In general, chemically distinct probes are bound to the different features of an array, so that each feature corresponds to a particular nucleotide sequence. In FIGS. 4-6, the principle of array-based hybridization assays is illustrated with respect to the single feature 404 to which a number of identical probes 405-409 are bound. In practice, each feature of the array contains a high density of such probes but, for the sake of clarity, only a subset of these are shown in FIGS. 4-6.

Once an array has been prepared, the array may be exposed to a sample solution of target DNA or RNA molecules (410-413 in FIG. 4) labeled with fluorophores, chemoluminescent compounds, or radioactive atoms 415-418. Labeled target DNA or RNA hybridizes through base pairing interactions to the complementary probe DNA, synthesized on the surface of the array. FIG. 5 shows a number of such target molecules 502-504 hybridized to complementary probes 505-507, which are in turn bound to the surface of the array 402. Targets, such as labeled DNA molecules 508 and 509, that do not contains nucleotide sequences complementary to any of the probes bound to array surface do not hybridize to generate stable duplexes and, as a result, tend to remain in solution. The sample solution is then rinsed from the surface of the array, washing away any unbound labeled DNA molecules. Finally, as shown in FIG. 6, the bound labeled DNA molecules are detected via optical or radiometric scanning. Optical scanning involves exciting labels of bound labeled DNA molecules with electromagnetic radiation of appropriate frequency and detecting fluorescent emissions from the labels, or detecting light emitted from chemoluminescent labels. When radioisotope labels are employed, radiometric scanning can be used to detect the signal emitted from the hybridized features. Additional types of signals are also possible, including electrical signals generated by electrical properties of bound target molecules, magnetic properties of bound target molecules, and other such physical properties of bound target molecules that can produce a detectable signal. Optical, radiometric, or other types of scanning produce an analog or digital representation of the array as shown in FIG. 7, with features to which labeled target molecules are hybridized similar to 706 optically or digitally differentiated from those features to which no labeled DNA molecules are bound. In other words, the analog or digital representation of a scanned array displays positive signals for features to which labeled DNA molecules are hybridized and displays negative features to which no, or an undetectably small number of, labeled DNA molecules are bound. Features displaying positive signals in the analog or digital representation indicate the presence of DNA molecules with complementary nucleotide sequences in the original sample solution. Moreover, the signal intensity produced by a feature is generally related to the amount of labeled DNA bound to the feature, in turn related to the concentration, in the sample to which the array was exposed, of labeled DNA complementary to the oligonucleotide within the feature.

Array-based hybridization techniques allow extremely complex solutions of DNA molecules to be analyzed in a single experiment. An array may contain from hundreds to tens of thousands of different oligonucleotide probes, allowing for the detection of a subset of complementary sequences from a complex pool of different target DNA or RNA polymers. In order to perform different sets of hybridization analyses, arrays containing different sets of bound oligonucleotides are manufactured by any of a number of complex manufacturing techniques. These techniques generally involve synthesizing the oligonucleotides within corresponding features of the array through a series of complex iterative synthetic steps.

An array may include any one-, two- or three-dimensional arrangement of addressable regions, called “features,” bearing a particular chemical moiety or moieties, such as biopolymers, associated with that region. Array features are typically, but need not be, separated by intervening spaces. Array features contain probe molecules or other chemical entities bound to the array substrate. The probes are designed or selected to bind to target molecules or other chemical entities in sample solutions.

Any given array substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, square features may have widths, or round feature may have diameters, in the range from a 10 μm to 1.0 cm. In other embodiments each feature may have a width or diameter in the range of 1.0 μm to 1.0 mm, usually 5.0 μm to 500 μm, and more usually 10 μm to 200 μm. At least some, or all, of the features may be of different compositions (for example, when any repeats of each feature composition are excluded the remaining features may account for at least 5%, 10%, or 20% of the total number of features). Interfeature areas are typically, but not necessarily, present. Interfeature areas generally do not carry probe molecules. Such interfeature areas typically are present where the arrays are formed by processes involving drop deposition of reagents, but may not be present when, for example, photolithographic array fabrication processes are used. When present, interfeature areas can be of various sizes and configurations.

Each array may cover an area of less than 100 cm², or even less than 50 cm², 10 cm² or 1 cm². In many embodiments, the substrate carrying the one or more arrays will be shaped generally as a rectangular solid having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. Other shapes are possible, as well. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein. Also, instead of drop deposition methods, photolithographic array fabrication methods may be used such as described in U.S. Pat. No.5,599,695, U.S. Pat. No.5,753,788, and U.S. Pat. No. 6,329,143. Interfeature areas need not be present particularly when the arrays are made by photolithographic methods as described in those patents.

A molecular array is typically exposed to a sample including labeled target molecules, and the array is then read. Reading of the array may be accomplished by illuminating the array and reading the location and intensity of resulting fluorescence at multiple regions on each feature of the array. For example, a scanner may be used for this purpose, which is similar to the AGILENT MICROARRAY SCANNER manufactured by Agilent Technologies, Palo Alto, Calif. Other suitable apparatus and methods are described in U.S. patent applications: Ser. No. 10/087447 “Reading Dry Chemical Arrays Through The Substrate” by Corson et al., and Ser. No. 09/846125 “Reading Multi-Featured Arrays” by Dorsel et al. As previously mentioned, these references are incorporated herein by reference. However, arrays may be read by any other method or apparatus than the foregoing, with other reading methods including other optical techniques, such as detecting chemiluminescent or electroluminescent labels, or electrical techniques, for where each feature is provided with an electrode to detect hybridization at that feature in a manner disclosed in U.S. Pat. No. 6,251,685, U.S. Pat. No. 6,221,583 and elsewhere.

A result obtained from the reading followed by application of a method of the present invention, may be used in that form or may be further processed to generate a result such as that obtained by forming conclusions based on the pattern read from the array (such as whether or not a particular target sequence may have been present in the sample, or whether or not a pattern indicates a particular condition of an organism from which the sample came). A result of the reading (whether further processed or not) may be forwarded (such as by communication) to a remote location if desired, and received there for further use (such as further processing). When one item is indicated as being “remote” from another, this is referenced that the two items are at least in different buildings, and may be at least one mile, ten miles, or at least one hundred miles apart. “Communicating” information references transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data.

As pointed out above, array-based assays can involve other types of biopolymers, synthetic polymers, and other types of chemical entities. A biopolymer is a polymer of one or more types of repeating units. Biopolymers are typically found in biological systems and particularly include polysaccharides, peptides, and polynucleotides, as well as their analogs such as those compounds composed of, or containing, amino acid analogs or non-amino-acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. For example, a “biopolymer” includes DNA (including cDNA), RNA, oligonucleotides, and PNA and other polynucleotides as described in U.S. Pat. No. 5,948,902 and references cited therein, regardless of the source. An oligonucleotide is a nucleotide multimer of about 10 to 100 nucleotides in length, while a polynucleotide includes a nucleotide multimer having any number of nucleotides.

As an example of a non-nucleic-acid-based molecular array, one might attach protein antibodies to features of the array that would bind to soluble labeled antigens in a sample solution. Many other types of chemical assays may be facilitated by array technologies. For example, polysaccharides, glycoproteins, synthetic copolymers, including block coploymers, biopolymer-like polymers with synthetic or derivitized monomers or monomer linkages, and many other types of chemical or biochemical entities may serve as probe and target molecules for array-based analysis. A fundamental principle upon which arrays are based is that of specific recognition, by probe molecules affixed to the array, of target molecules, whether by sequence-mediated binding affinities, binding affinities based on conformational or topological properties of probe and target molecules, or binding affinities based on spatial distribution of electrical charge on the surfaces of target and probe molecules.

Scanning of a molecular array by an optical scanning device or radiometric scanning device generally produces a scanned image comprising a rectilinear grid of pixels, with each pixel having a corresponding signal intensity. These signal intensities are processed by an array-data-processing program that analyzes data scanned from an array to produce experimental or diagnostic results which are stored in a computer-readable medium, transferred to an intercommunicating entity via electronic signals, printed in a human-readable format, or otherwise made available for further use. Molecular array experiments can indicate precise gene-expression responses of organisms to drugs, other chemical and biological substances, environmental factors, and other effects. Molecular array experiments can also be used to diagnose disease, for gene sequencing, and for analytical chemistry. Processing of molecular array data can produce detailed chemical and biological analyses, disease diagnoses, and other information that can be stored in a computer-readable medium, transferred to an intercommunicating entity via electronic signals, printed in a human-readable format, or otherwise made available for further use.

Two or more data sets can be obtained from a single molecular array by scanning the molecular array for two or more signals. When optical scanning is used to detect fluorescent or chemiluminescent emission from chemophore labels, a first signal, or data set, may be generated by scanning the molecular at a first optical wavelength, and a second signal, or data set, may be generated by scanning the molecular at a second optical wavelength. Different signals may be obtained from a molecular array by radiometric scanning two detect radioactive emissions at two different energy levels. Target molecules may be labeled with either a first chromophore that emits light at a first wavelength, or a second chromophore that emits light at a second wavelength. Following hybridization, the molecular array can be scanned at the first wavelength to detect target molecules, labeled with the first chromophore, hybridized to features of the molecular array, and can then be scanned at the second wavelength to detect target molecules, labeled with the second chromophore, hybridized to the features of the molecular array. In one common molecular array system, the first chromophore emits light at a red visible-light wavelength, and the second chromophore emits light at a green, visible-light wavelength. The data set obtained from scanning the molecular array at the red wavelength is referred to as the “red signal,” and the data set obtained from scanning the molecular array at the green wavelength is referred to as the “green signal.” While it is common to use two different chromphores, it is possible to use three, four, or more different chromophores and to scan a molecular array at three, four, or more wavelengths to produce three, four, or more data sets.

The ability to use different chromophores and to scan a molecular array at different wavelengths allows researchers to employ a single molecular array to determine the presence and concentrations of target molecules in multiple sample solutions. For example, in gene-expression experiments, the cDNA fragments produced from a solution of mRNAs extracted from a tissue sample at time t₁ can be labeled with a first chromophore to produce a first sample solution, and cDNA fragments produced from a solution of mRNA molecules extracted from the tissue sample at time t₂ can be labeled with a second chromophore to produce a second sample solution. A molecular array can be simultaneously exposed to the first and second sample solutions and then scanned at the first and second wavelengths in order to produce data sets reflective of gene-expression levels within the tissue at times t₁ and t₂.

Unfortunately, the red and green data sets are not directly comparable. The relative magnitudes of the individual red and green feature signals, or signal intensities measured from individual features, are not directly proportional to the relative concentrations of mRNA molecules in the tissue samples. There may be differences in labeling efficiencies for the red and green chromophores, for example. There may be differences in the strength of the red and green signals produced by the same quantity, or concentration, of red and green chromophores within a feature. For example, light emitted by one chromophore may be more readily reabsorbed by surrounding molecules than light emitted by the other chromophore. The spectral response of the molecular array scanner may differ for the two different wavelengths of light. For these, and many additional reasons, the ratios of the red to green signals for the features of the molecular array are not directly proportional to the ratios of the concentrations of the labeled target molecules in the two different sample solutions.

In order to compare the red and green signals, the red and green data sets must be normalized. Currently, data normalization is generally carried out by determining a global ratio of red to green signals for a subset of features containing probe molecules directed to so-called housekeeping genes, believed to be uniformly expressed over the course of the experiments, mRNAs for which are therefore present at similar concentrations in both sample solutions being analyzed. One data set is then rescaled to the other, employing the global ratio. However, it is becoming clear that the so-called housekeeping genes are, in fact, often expressed at different levels at different times and within different tissues. Thus, the assumption that a particular subset of genes is consistently expressed at constant levels over the course of multiple experiments has shown to be inaccurate. Other types of global normalization methods, such as division of the feature-signal magnitude of a data set by the geometric mean for the feature-signal magnitudes and curve-fitting the resulting globally normalized data according to various mathematical models have been employed, but suffer from the failure of the mathematical models to take into account the many different parameters that may contribute to different types of variations observed in the signals produced by scanning molecular arrays. Moreover, many of the current techniques are computationally complex. For these reasons, designers and manufacturers of molecular arrays and molecular array scanners, as well as researchers and other users of molecular arrays, have recognized the need for a computationally efficient method and system for accurately normalizing data sets collected from molecular arrays without relying on assumptions concerning the expression levels for certain genes or on assumptions about the various factors and parameters that produce variability in data sets.

### SUMMARY OF THE INVENTION

One embodiment of the present invention provides a method and system for normalizing two molecular array data sets. In this embodiment, the two data sets are both separately globally normalized by, for example, dividing the feature-signal magnitudes of each data set by the geometric mean of the feature-signal magnitudes of the data set. Alternatively, the arithmetic mean of the feature-signal magnitudes, or other distribution metrics, may be employed for globally normalizing both data sets. Next, the globally normalized feature signal magnitudes within each data set are ranked in ascending order. Following the ranking step, a numeric function is created that relates feature-signal magnitudes of one data set to corresponding feature-signal magnitudes of the other data set with respect to feature-signal magnitude. Only a subset of the features is used to construct the numeric function. This feature subset is obtained by selecting features that are similarly ranked in the separate feature-signal-magnitude rankings for the two data sets. This subset of features corresponds to those features associated with feature-signal magnitudes that represent a central trend in both data sets. In one embodiment, the numeric function comprises data points representing the ratio of globally normalized signal magnitudes for a particular feature plotted as a function of the globally normalized feature-signal magnitudes of one of the two data sets. Next, the numeric function is smoothed by one of many possible different smoothing procedures. The ratio of feature-signal magnitudes for a given feature can then be obtained from the smoothed numeric function and used to rescale the feature-signal magnitude in one data set to the feature-signal magnitude in the other data set to produce a final, normalized pair of data sets.

The number of differently ranked members in any one of the feature signal-magnitude data sets can, for example, be at least 100, 200, or at least 1000, and the number of selected normalizing features can, for example, be at least 50, 100, 200 or 500.

In an alternate embodiment, the locally weighted least squares smoothing method (“LOWESS”) is used to smooth the numeric function, and also employed to calculate the ratio of globally normalized signal magnitudes for a particular feature based on a combined signal magnitude of that feature. In the alternate embodiment, rather than rescaling one data set to another, both data sets are concurrently rescaled by distributing a LOWESS correction, or weight, between the normalized signal magnitudes of the data sets.

The described embodiments of the present invention can be employed to normalize data sets corresponding to multiple signals scanned from a single molecular array, and can also be applied to normalize molecular array data sets obtained from different molecular arrays. The method and system can be straightforwardly extended in order to normalize more than two data sets. The described embodiments of the present invention are computationally efficient, and do not assume consistent expression levels for particular genes nor particular mathematical models for the variations within and between data sets.

The method of the present invention can be implemented as part of a computer program product that normalizes molecular array data sets. The computer program product may include a computer readable storage medium from which the computer program can be loaded into a computer memory and executed by a computer processor to carry out various embodiments of the method of the present invention.

### BRIEF DESCRIPTION OF THE DRAWINGS

FIG. 1 illustrates a short DNA polymer.

FIG. 2A shows hydrogen bonding between adenine and thymine bases of corresponding adenosine and thymidine subunits.

FIG. 2B shows hydrogen bonding between guanine and cytosine bases of corresponding guanosine and cytosine subunits.

FIG. 3 illustrates a short section of a DNA double helix.

FIGS. 4-7 illustrate the principle of array-based hybridization assays.

FIG. 8 shows two, very small, example molecular-array data sets.

FIG. 9 shows the hypothetical feature-signal data sets of FIG. 8 following global normalization.

FIG. 10 shows linear, 1-dimensional arrays corresponding to the 2-dimensional arrays of FIG. 9.

FIG. 11 shows sorting arrays corresponding to the arrays containing the globally normalized data sets shown in FIG. 10.

FIG. 12 shows the sorting arrays shown in FIG. 11 following a sorting procedure.

FIGS. 13A-E illustrate the feature selection process used to construct the normalizing feature subset.

FIG. 14 shows the numeric function array following a complete traversal of the sorting arrays for the hypothetical data set stored in the 1-dimensional array show in FIG. 10.

FIG. 15 shows a standard, 2-dimensional graphical plot of the RTF represented by the data points instead of the numeric array illustrated in FIG. 14.

FIG. 16 shows the numeric function represented by data points in the numeric function array of FIG. 14 and plotted in FIG. 15 following a smoothing process.

FIG. 17 shows RTF array that includes the data points for all features in the C1 and C2 data sets determined from the smoothed RTF by this method.

FIG. 18 shows the renormalized data sets following rescaling of the \overline{C2} data set using the RTF function.

FIG. 19 shows a plot of an RTF obtained from self-comparison experiments.

FIGS. 20A-B, show the log ratios calculated for normalized data sets generated from a yeast-mRNA self-comparison experiment.

FIG. 21 shows RTFs obtained from data sets generated by various differential gene-expression comparison experiments.

FIGS. 22A-B show plots of log expression ratios obtained from gene-expression differential comparison experiments with respect to net normalized feature-signal magnitudes.

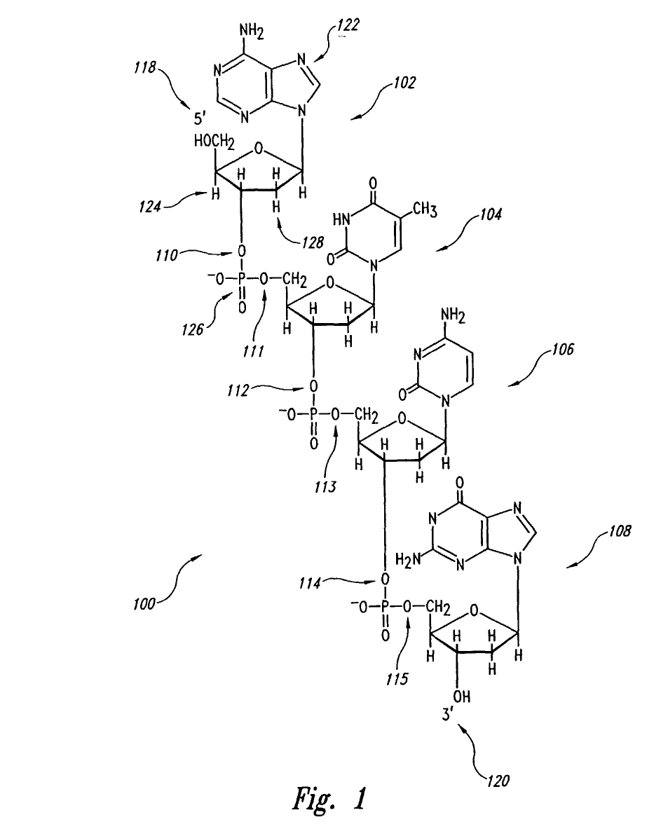

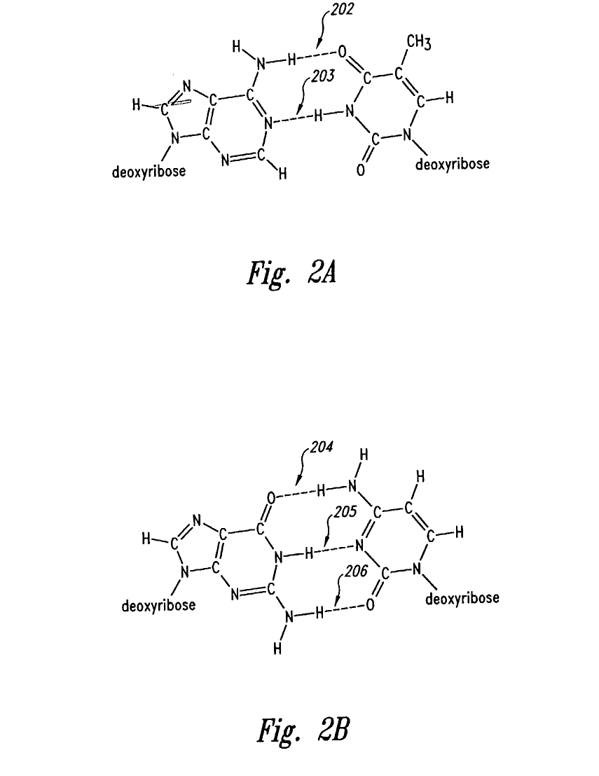

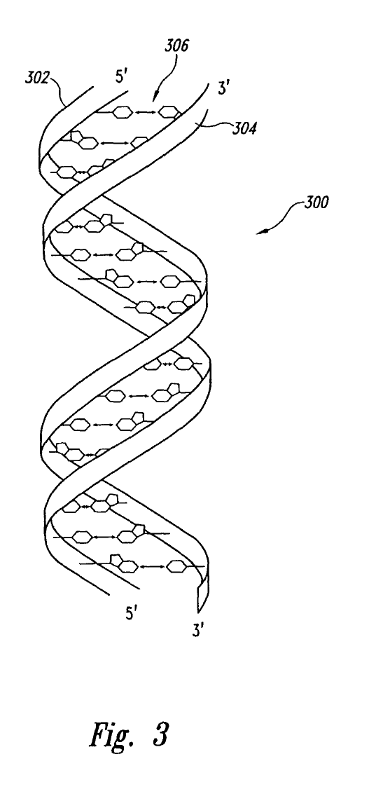

### DETAILED DESCRIPTION OF THE INVENTION

One embodiment of the present invention is directed to a method and system for normalizing molecular array data sets. One embodiment of the present invention employs a local normalization technique that makes no assumptions about the relative magnitudes of signals in two or more data sets corresponding to a particular feature. Moreover, the method does not rely on closed-form mathematical models for variations in signal magnitudes within, and in between, data sets. However, the normalization methods are based on the assumption that a substantial subset of features of the molecular array contains probes directed to target molecules that have similar concentrations in the sample solutions to which the molecular array is exposed. In gene-expression experiments, this assumption is equivalent to the more specific assumption that a substantial portion of the genes corresponding to molecular-array probes are expressed at relatively constant levels throughout the experiments that generate the data sets to be normalized. The disclosed embodiments are computationally efficient both with respect to run-time operation as well as with respect to initial development and testing, and have been shown to produce reliable and accurate normalization in various self-comparison and differential expression experiments.

It should be noted that embodiments of the method of the present invention may be incorporated into molecular array scanners or other instruments, and may also be incorporated into computer programs that process molecular array data. The data obtained from a molecular array, and processed by a computer program, may be stored or encoded in a variety of electronic, magnetic, optical, and other computer readable and electronically transferable forms and transmitted over great distances.

Various embodiments of the present invention are described, below, in three subsections. In the first subsection, a first embodiment is described with reference to a series of figures that illustrate normalization of two, very small, hypothetical data sets. In the first subsection, a number of mathematical formulas are provided to concisely describe the theoretical basis for the first embodiment of the present invention. In a second subsection, a concise, C++-like pseudocode implementation of the first embodiment of the normalization method is provided. This pseudocode is described in detail in the associated text. In a third subsection, various alternative embodiments and improvements on the basic method and system described in the first two subsections are provided.

It should be noted that the following descriptions are generally based on gene-expression experiments. However, the normalization methods that represent several embodiments of the present invention may be employed to normalize data sets obtained from molecular-array-based experiments involving analysis of target molecules other than mRNAs or other direct or indirect gene products. The only assumption, as discussed above, is that a substantial portion of the target molecules comprises target molecules that are present at relatively constant levels in the sample solutions from which the data sets are generated.

A Graphical and Mathematical Description of a First Embodiment of the Method of the Present Invention

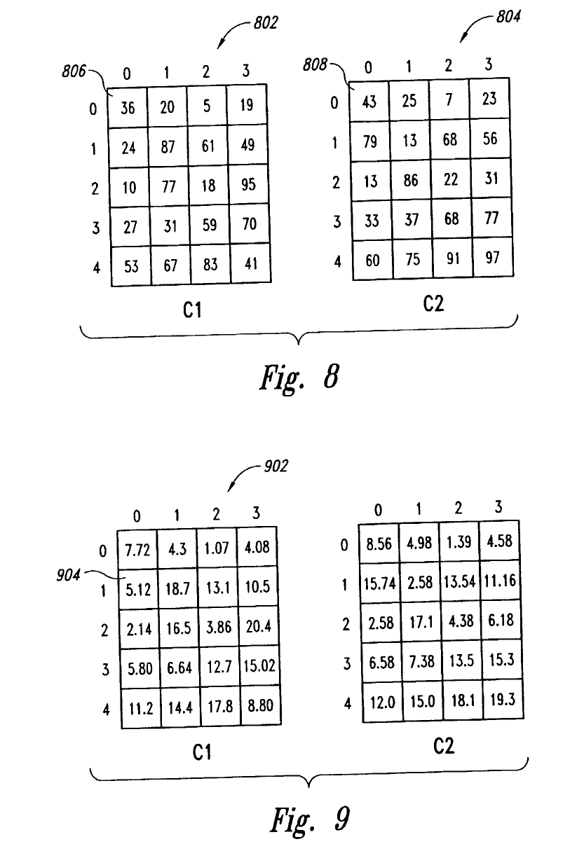

FIG. 8 shows two, very small, example molecular-array data sets. A first array 802 contains the signal magnitudes of a small number of features obtained by scanning a molecular array at a wavelength corresponding to the color “C1.” A second array 804 contains the magnitudes of signals obtained from the same features by scanning the molecular array at a different wavelength corresponding to the color “C2.” The feature-signal magnitudes in the first array 802 thus comprise the C1 data set, and the feature-signal magnitudes in the second array 804 thus comprise the C2 data set. Thus, for example, feature C1(0, 0) produced a signal of magnitude “36” 806 when scanned at wavelength “C1” and produced a signal of magnitude “43” 808 when scanned at wavelength “C2.” Note that, in general, there appears to be a correlation between the two data sets. For example, features C1(0, 0), C1(0, 1), C1(0, 2), and C1(0, 3), in the first row of the first array 802, have signal magnitudes “36,” “20,” “5,” and “19,” respectively, while features C2(0, 0), C2(0, 1), C2(0, 2), and C2(0, 3), in the first row of the second array 804 have signal magnitudes “43,” “25,” “7,” and “23,” respectively. Thus, considering the first row of both data sets, the relative magnitudes of the corresponding features appear to be relatively equivalent, with the feature-signal magnitudes in data set C2 somewhat larger than the corresponding feature-signal magnitudes in data set C1. However, it is difficult to know whether the differences in the magnitudes of the signals for corresponding features in the two data sets reflect difference in the concentrations of the corresponding labeled target molecules in the two different samples to which a molecular array was exposed, or whether the differences in feature-signal magnitudes represent signal-magnitude variations due to different labeling efficiencies, different reabsorption of emitted light from different chromophores, or different spectral responses of scanner detectors, among other possible variations, that should be corrected by a normalization process. Note, too, that the feature signal magnitudes for a small number of features are quite different in the two data sets. For example, consider features C1(1, 0), C1(1, 1) C2(1, 0), and C2(1, 1). The C1-data-set features have values “24” and “87,” respectively, while the C2-data-set features have values “79” and “13,” respectively.

A first step of one embodiment of the present invention is to globally, and independently, normalize the feature-signal magnitudes in the two data sets. Many alternative methods may be employed for this initial, global normalization. In one method, each measured, raw feature-signal magnitude is divided by a distribution metric based on the magnitudes of all the features signals of the data set. One distribution metric is the artimethtic mean. The arithmetic mean can be expressed as:

$$( {\sum }_{i = 1}^{N} {w}_{i} {s}_{i} ) / {\sum }_{i = 1}^{N} {w}_{i}$$

where N is the number of feature signals in the data set;

wᵢ is the weight assigned to the signal measured for feature i; and

sᵢ is the magnitude of the signal measured for feature i.

In general, the feature-signal data sets are first processed to detect anomalous signals, or outliers, which are omitted from subsequent calculations. Details of outlier detection, background subtraction, and other preprocessing operations can be found in U.S. patent application Ser. Nos: 09/589,046 filed Jun. 6, 2000 and 10/086,839 filed Feb. 28, 2002. Thus, the weight wᵢ for an outlier feature is 0, while the weight wᵢ for a non-outlier, or valid feature is 1. Various different weighting schemes may be employed in order to take into account additional categories of features detected during initial processing in addition to outlier features and non-outlier features. A second, commonly employed distribution metric is the geometric mean, provided by the following formula:

$$anti {log}_{10} [ ( {\sum }_{i = 1}^{N} {w}_{i} {log}_{10} ( {s}_{t} ) ) / {\sum }_{i = 1}^{N} {w}_{i} ]$$

where N, wᵢ, and sᵢ have the same meanings as in the previous expression defining the arithmetic mean.

The arithmetic mean used in the normalization method is the average value of valid feature signal magnitudes obtained by adding the valid feature signal magnitudes together and dividing the result by the total number of feature-signal magnitudes added together, while the geometric mean is the nth root of the value obtained by multiplying together all the valid feature-signal magnitudes.

FIG. 9 shows the hypothetical feature-signal data sets of FIG. 8 following global normalization. In FIG. 9, the globally normalized signal magnitudes for each feature are obtained by dividing the raw signal magnitude corresponding to the feature by the arithmetic mean of the raw signal magnitudes of the data set, and then multiplying by 10. The globally normalized data sets are referred to as “\overline{C1}” and “\overline{C2},” and globally normalized feature-signal magnitudes are referred to as “\overline{sᵢ}.” These values have units referred to as deciNorms. A signal magnitude divided by an arithmetic or geometric mean is referred to in terms of the unit “Norm.” Thus, when a signal magnitude has the same value as the average of the signal magnitudes, the normalized signal magnitude is 1 Norm. When a signal magnitude has a value twice as great as the average of the signal magnitudes, the normalized signal magnitude is 2 Norm. A deciNorm is equivalent to 1/10 of a Norm, a centiNorm is equivalent to 1/100 of a Norm, and a milliNorm is equivalent to 1/1000 of a Norm. CentiNorms and milliNorms are commonly employed units for globally normalized feature-signal magnitudes.

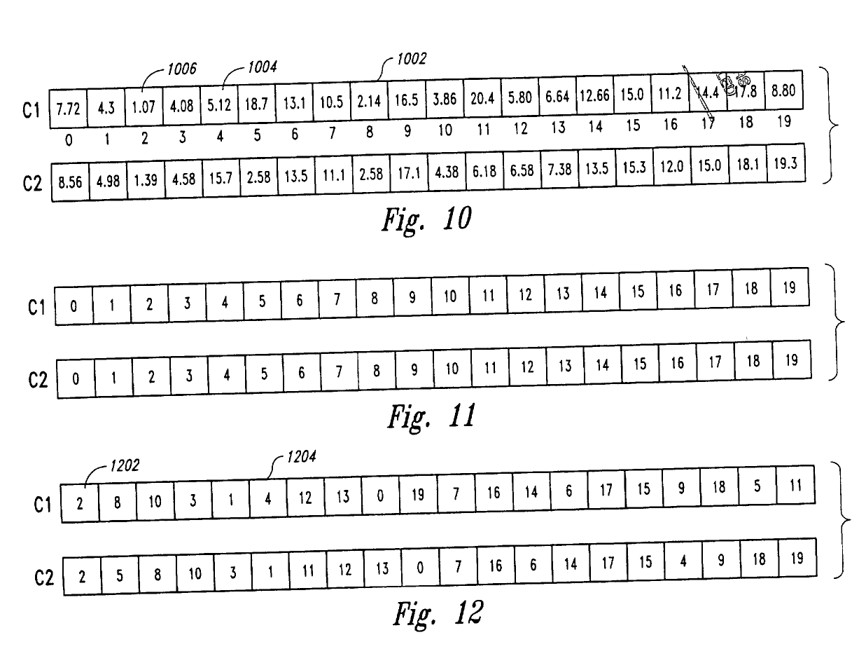

While it is convenient to store, and refer to, raw data in 2-dimensional matrices corresponding to the 2-dimensional feature grids of molecular arrays, as shown in FIGS. 8-9, it is desirable to store, and refer to, data in 1-dimensional arrays for subsequent calculations. FIG. 10 shows linear, 1-dimensional arrays corresponding to the 2-dimensional arrays of FIG. 9. In FIG. 10, the 2-dimensional arrays of FIG. 9 have been linearized by adding each successive row of the 2-dimensional arrays to the 1-dimensional arrays. Thus, for example, the element 1004 with index “4” in the 1-dimensional array 1002 in FIG. 10 corresponds to the element \overline{C1} (1, 0) 904 in the 2-dimensional array 902 representing data set \overline{C1} in FIG. 9.

In a next step in the normalization process that represents one embodiment of the present invention, two additional 1-dimensional sorting arrays are initialized to contain the indices of the elements in the 1-dimensional arrays that contain the globally normalized data sets shown in FIG. 10. FIG. 11 shows the sorting arrays corresponding to the arrays containing the globally normalized data sets shown in FIG. 10. Next, the indices in the sorting array are sorted in ascending order with respect to the values, contained in the 1-dimensional normalized data-set arrays, indexed by the indices. FIG. 12 shows the sorting arrays shown in FIG. 11 following a sorting procedure. Note, for example, that the first index stored in the first element 1202 of the first sorting array 1204 following sorting corresponds to the index of the element 1006 of the first, globally normalized data-set array 1002 which contains the smallest normalized feature-signal magnitude. The sorting arrays shown in FIG. 12 represent a rank ordering of the globally normalized data sets. Any number of different, well-known sorting techniques can be employed to sort the sorting arrays based on the feature-signal magnitudes.

In a next step, features are selected to compose a subset of normalizing features with signal magnitudes that represent a central trend common to both data sets. A feature is selected for the subset of normalizing features if the signal magnitude of the feature in both data sets is similarly ranked. In other words, referring to the sorting array shown in FIG. 12, if the index for a feature is contained in similarly indexed elements in the sorting arrays, or, in other words, has a similar, relative position within both sorting arrays, then the feature is selected for the subset.

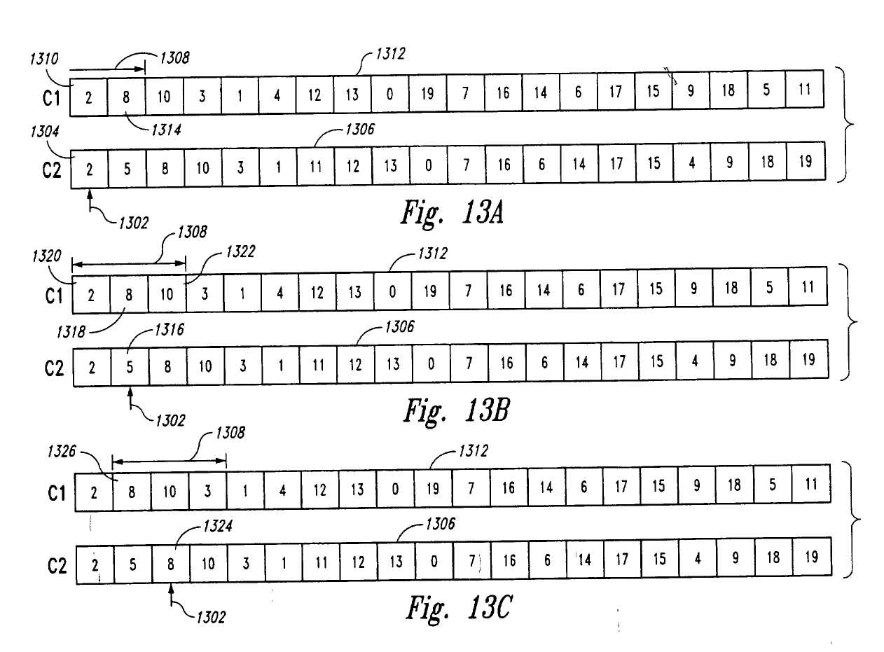

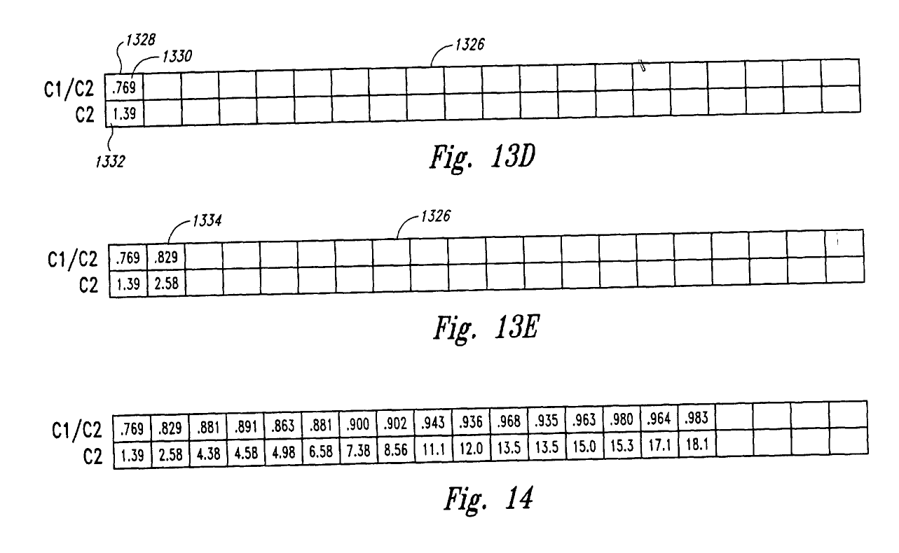

FIGS. 13A-E illustrate the feature selection process used to construct the normalizing feature subset. For the example illustrated in the FIGS. 8-18, a sliding window spanning three elements in one sorting array, and a corresponding element pointer in the other sorting array, are employed. The sliding window is defined in terms of a number of elements, in this simple example, but may be defined in terms of a range of feature-signal magnitudes, or another metric that restricts the range of elements in one sorting array that are searched for an index corresponding to an index located in the element referenced by the element pointer in the other sorting array. Initially, at the start of the process, the window spans only two elements, as the window is generally symmetric about the sorting array index of the element referenced by the element pointer in the other sorting array.

FIG. 13A illustrates the initial step of the normalizing feature selection process. An element pointer 1302 is initialized to point of the first element 1304 of the sorting array 1306 for the \overline{C2} data set. An initial, partial sliding window 1308 is constructed relative to the corresponding element 1310 in the sorting array 1312 for the \overline{C1} data set. In this first step, the elements 1310 and 1314 spanned by the initial sliding window 1308 are searched for an element containing the index contained in the element 1304 referenced by the element pointer 1302. If the index “2” is found within the elements 1310 and 1314 spanned by the sliding window 1308, then the feature indexed by the index “2” is selected for the feature subset. If, by contrast, the index “2” is not found within the elements spanned by the sliding window, then the feature indexed by the index “2” is not selected for the feature subset. In the present case, the element 1310 contains the index “2,” and thus the feature indexed in the 1-dimensional arrays containing the globally normalized data sets, shown in FIG. 10, with index “2” is selected for the subset of normalizing features.

In the next step of the feature selection process, illustrated in FIG. 13B, the element pointer 1302 is incremented to the point to the next element 1316 in the sorting array 1306 for the \overline{C2} data set. The sliding window 1308 is moved rightward by one element along the sorting array 1312 for the \overline{C1} data set. Note that the sliding window now spans three elements, and is symmetrical about the element 1318 of the sorting array 1312 for the \overline{C1} data set that corresponds to the element 1316 of the sorting array 1306 for the \overline{C2} data set referenced by the element pointer 1302. The elements 1318, 1320, 1322 spanned by the sliding window 1308 are then searched for an element containing the index “5” containing element 1316 referenced by the element pointer 1302. In this case, no such element is found. Therefore, the feature indexed by index “5” in the 1-dimensional arrays show in FIG. 10 is not selected for the normalizing feature subset.

FIG. 13C shows the next, successive step of the feature selection process. In FIG. 13C, the element pointer 1302 has been incremented to point to the next element 1324 in the sorting array 1306 for the \overline{C2} data set. The sliding window 1308 has also been moved rightward by one element along the sorting array 1312 for the \overline{C1} data set. Again, the elements spanned by the sliding window 1308 are searched for an element containing the index “8,” contained in the element 1324 referenced by the element pointer 1302. In this case, the element 1326 is found to contain the index “8,” and the feature indexed by index “8” in the 1-dimensional array shown in FIG. 10 is thus selected for inclusion in the feature subset. In additional steps not illustrated in the figures, the entire \overline{C2} sorting array is traversed by the element pointer, and the \overline{C1} sorting array is traversed by the sliding window, and the remaining features are thus evaluated for inclusion in the normalizing feature subset.

Another approach to selecting the normalizing features can be expressed mathematically by computing a normalized rank fractional distance δᵢ for each feature i and selecting as normalizing features only those features with normalized rank fractional distances less than a threshold value. A normalized rank fractional distance is computed as:

δᵢ=|rank(\overline{s}ᵢ_{_{C2}})−rank(\overline{s}ᵢ_{_{C1}})|/N_{valid}

where \overline{s}ᵢ_{_{C2}} is the globally normalized signal magnitude for feature i in the \overline{C2} data set,

\overline{s}ᵢ_{_{C1}} is the globally normalized signal magnitude for feature i in the \overline{C1} data set,

rank(s) is the rank is the rank of a globally normalized feature-signal magnitude in its corresponding sorting array, and

N_{valid} is the number of valid, or ranked features, or, in other words, the number of features in the union of the sets of outlying features in data sets C1 and C2 subtracted from the total number of features N.

The sliding-window method is, in fact, equivalent to the normalized rank fractional distance method, where the extent, or size, of the sliding window is derived from the δᵢ threshold.

The normalizing features are next used to construct a numeric function, referred to as the ratio transfer function (“RTF”), that relates the ratios of normalized signal magnitudes for each feature of the normalizing features to the signal magnitudes of the normalizing features in one data set. In the currently described embodiment, the RTF can be expressed as:

ƒ_{RTF}(\overline{s}ᵢ_{_{C1}})=\overline{s}ᵢ_{_{C1}}/\overline{s}ᵢ_{_{C2}}

where \overline{s}ᵢ_{_{C1}} and \overline{s}ᵢ_{_{C2}} have the same meanings as in the previous equation.

FIGS. 13D and 13E illustrate the contents of an array containing sample points for the RTF that is constructed based on the globally normalized signal magnitudes of the features selected for the normalizing feature subset. The domain for this RTF, as shown above, is the globally normalized signal magnitudes for the normalizing features in the \overline{C2} data set. The range for this RTF is the ratios of the globally normalized signal magnitudes for the normalizing features in the \overline{C1} and \overline{C2} data sets, as shown in the above expression.

FIG. 13D shows the contents of the RTF array 1326 following the first step of the feature subset selection process illustrated in FIG. 13A. In that step, the feature with index “2” is selected as the first normalizing feature, and a corresponding RTF data point is constructed for the feature according to the above expression and placed in the first element 1328 of the RTF array. The data point contains the ratio of the normalized signal magnitudes for the feature in the \overline{C1} and \overline{C2} data sets 1330 and the normalized signal magnitude for the feature in the \overline{C2} data set 1332. In the second step of the feature selection process, no feature is selected, so that, following the second step of the feature selection process, illustrated in FIG. 13B, the numeric function array continues to appear shown in FIG. 13B. Following the third step of the feature selection process, illustrated in FIG. 13C a second data point for the numeric function is constructed based on the signal magnitudes for the feature indexed by index “8” and placed into the second element 1334 of the numeric function array 1326.

As described above, the normalizing feature selection process continues in the manner suggested in FIGS. 13A-C, traversing the full length of the sorting arrays. For each selected feature, a data point is added to the numeric function array. FIG. 14 shows the numeric function array following a complete traversal of the sorting arrays for the hypothetical data set stored in the 1-dimensional array show in FIG. 10.

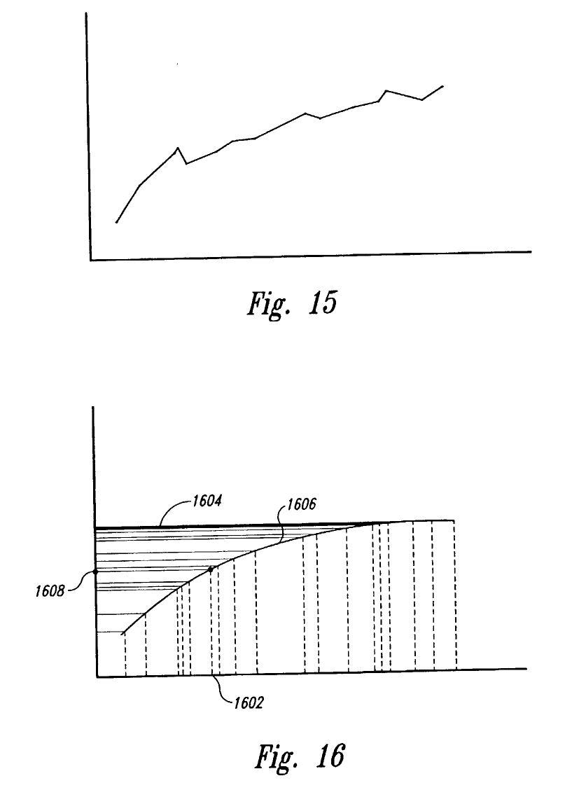

FIG. 15 shows a standard, 2-dimensional graphical plot of the RTF represented by the data points instead of the numeric array illustrated in FIG. 14. As can be seen in FIG. 15, the ratios of globally normalized \overline{C1} signal magnitudes to globally normalized signal magnitudes increases within increasing \overline{C2} signal magnitude. However, when the plotted data points are connected with straight lines, the RTF appears jagged, or discontinuous. The discontinuities can be attributed to various statistical errors and fluctuations in the data sets, as well as to the fact that only a subset of features are used to construct a discrete RTF.

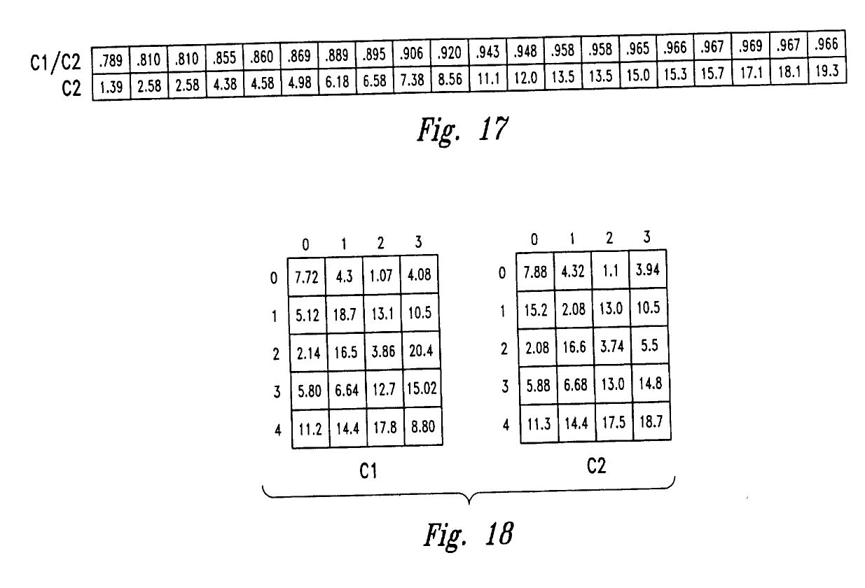

Discrete numeric functions, such as the RTF plotted in FIG. 15, can be mathematically smoothed to model a continuous curve normally produced by closed-form mathematical functions and normally observed for extensively sampled data sets generated from natural phenomenon following corrections for errors and statistically fluctuation. Many different, well-known smoothing techniques can be employed to transform the discrete RTF into an approximation of a continuous RTF, including linear regression and least-squared methods, local averaging methods, and kernel methods. In the third subsection, use of the LOWESS smoothing method is discussed. FIG. 16 shows the numeric function represented by data points in the numeric function array of FIG. 14 and plotted in FIG. 15 following a smoothing process. Once the discrete RTF has been smoothed, the data points for all features in the \overline{C2} data set can be estimated from the smooth function. For example, in FIG. 16, the \overline{C1}/\overline{C2} signal magnitude ratio for a feature with a particular \overline{C2} signal magnitude 1602 can be estimated by finding the point 1604 of intersection of a vertical line passing through point 1602 and the smoothed RTF 1606, and then determining the y-coordinate 1608 of the intersection point 1604. FIG. 17 shows RTF array that includes the RTF data points for all features in the C1 and C2 data sets determined from the smoothed RTF by this method.

In a final step, the \overline{C2} data set is renormalized, or rescaled, according to the smoothed RTF. Each signal magnitude of the \overline{C2} data set is multiplied by the \overline{C1}/\overline{C2} signal magnitude ratio determined for the \overline{C2} signal magnitude from the smoothed RTF. Thus, for a particular feature i, the rescaled signal magnitude \overline{s}ᵢ_{_{C2}} is given by the expression:

\overline{s}ᵢ_{_{C2}}=\overline{s}ᵢ_{_{C2}}ƒ_{RTF}(\overline{s}ᵢ_{_{C2}})

where \overline{s}ᵢ_{_{C1}} and \overline{s}ᵢ_{_{C2}} have the same meanings as in the previous equation.

FIG. 18 shows the renormalized data sets following resealing of the \overline{C2} data set using the RTF function. Note that, for most features, following renormalization, the renormalized signal magnitudes are nearly identical, while for the features, such as features C1(1, 0), C1(1, 1), C2(1, 0), and C2(1, 1), for which the signal magnitudes appear to be non-equivalent in the original data sets shown in FIG. 8, the renormalized signal magnitudes in the renormalized data sets shown in FIG. 18, continue to appear to be non-equivalent.

Along with producing renormalized data sets, the method that represents one embodiment of the present invention, described with respect to FIGS. 8-18, can also yield a numerical indication of the magnitude of the correction represented by the renormalization. One correction metric that can be applied is a root-mean-square of the individual corrections applied to signal magnitudes for the \overline{C2} data set:

$${[ {\sum }_{i = 1}^{{N}_{valid}} {( {f}_{RTF} ( {\overline{s}}_{{i}_{c2}} ) - 1 )}^{2} / {N}_{valid} ]}^{1 / 2}$$

More elaborate metrics may be devised, including a weighted root-mean-square where each term of the summation, in the above expression, is multiplied by a weighting factor. The weighting factor may be, in one alternative embodiment, a measure of the extent of the domain of the RTF represented by the estimated data point ƒ_{RTF}(\overline{s}ᵢ_{_{C2}}).

The normalized data sets are commonly used as the basis for additional computations, such as calculation of log expression ratios:

log₁₀(\overline{s}ᵢ_{_{C1}}/\tilde{s}ᵢ_{_{C2}})

Log expression ratios are commonly used to quantify differential gene expression as measured by 2-sample exposures of a molecular array.

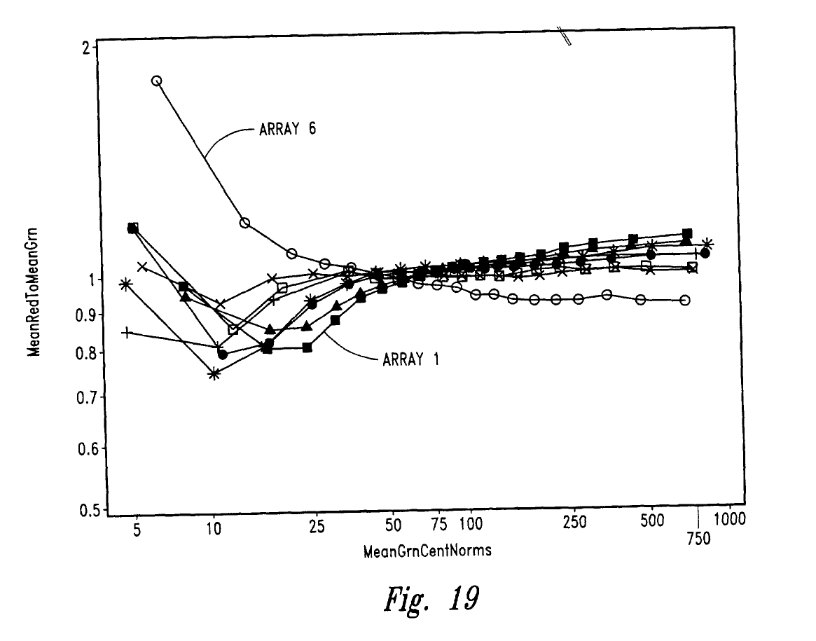

FIG. 19 shows a plot of several RTFs obtained from self-comparison experiments. In self-comparison experiments, a molecular array is exposed to two sample solutions containing identical concentrations of target molecules, in one sample solution labeled with a first chromophore, and in the other sample solution labeled with a second chromophore. As can be seen in FIG. 19, the RTFs for six self-comparison experiments are approximately equivalent, with one exception, and the RTFs tend to be relatively constant about the \overline{C1}/\overline{C2} ratio of 1.0, indicating relatively little correction required to renormalize the two data sets.

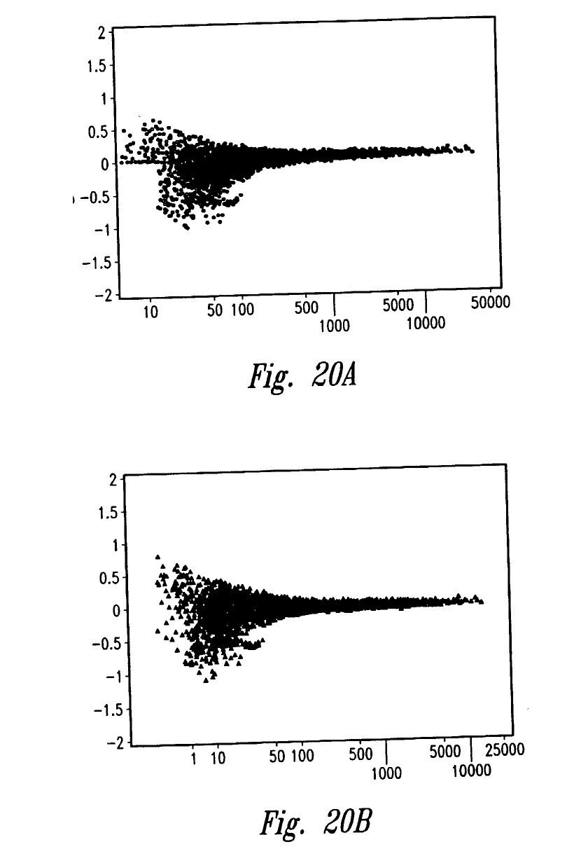

FIGS. 20A-B, show the log expression ratios calculated for normalized data sets generated from a yeast-mRNA self-comparison experiment plotted with respect to the globally normalized feature-signal magnitudes of one data set. In both FIGS. 20A and 20B, the log expression ratios are reasonably symmetrically distributed about the expected log ratio of 0.0 for a self-comparison experiment, and the variation in the distribution decreases with increasing signal magnitude. The data sets are normalized using a global normalization procedure to produce the log expression ratios shown in FIG. 20A. The data sets are normalized using an embodiment of the present localized normalization method to produce the log expression ratios shown in FIG. 20B. In FIG. 20A, the axis of symmetry of the distribution of log expression ratios is slightly tilted upward with increasing globally normalized feature-signal magnitude, while in FIG. 20B, the axis of symmetry of the distribution is horizontal. The tilting of the axis of symmetry in FIG. 20A, represents a systematic error arising for global normalization that is avoided by using the described embodiment of the present invention.

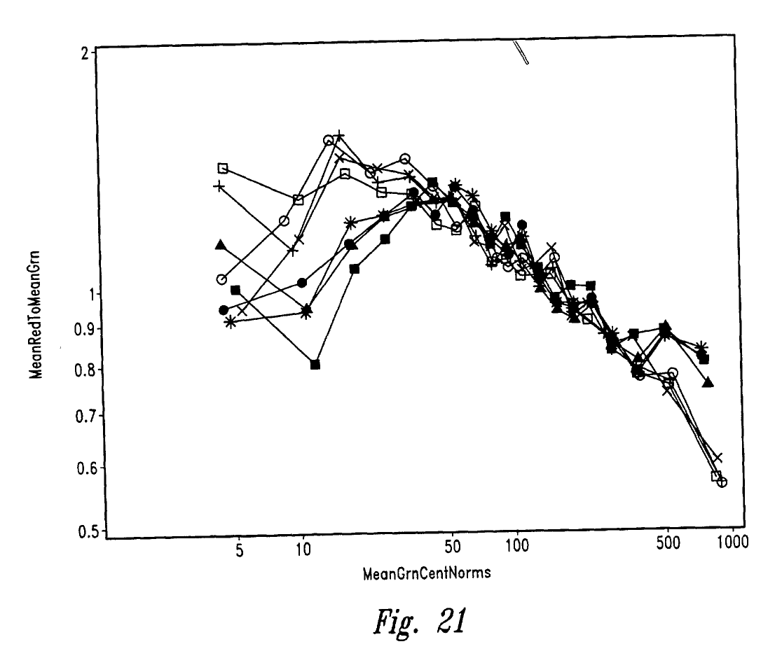

FIG. 21 shows RTFs obtained from data sets generated by various differential gene-expression comparison experiments. In FIG. 21, the RTFs plotted with solid symbols represent experiments in which target molecules of a first sample are labeled with a C1 chromophore, while target molecules of a second, different sample are labeled with a C2 chromophore. The open symbols represent experiments in which target molecules of a first sample are labeled with the C2 chromophore, while target molecules of a second, different sample are labeled with the C1 chromophore. In general, the size of the correction represented by the RTFs for differential comparison experiments are greater than for self-comparison experiments. As can be seen in FIG. 21, the RTFs into two distinct groups, one group generated by experiments in which target molecules of a first sample are labeled with the C1 chromophore, while target molecules of a second, different sample are labeled with the C2 chromophore.

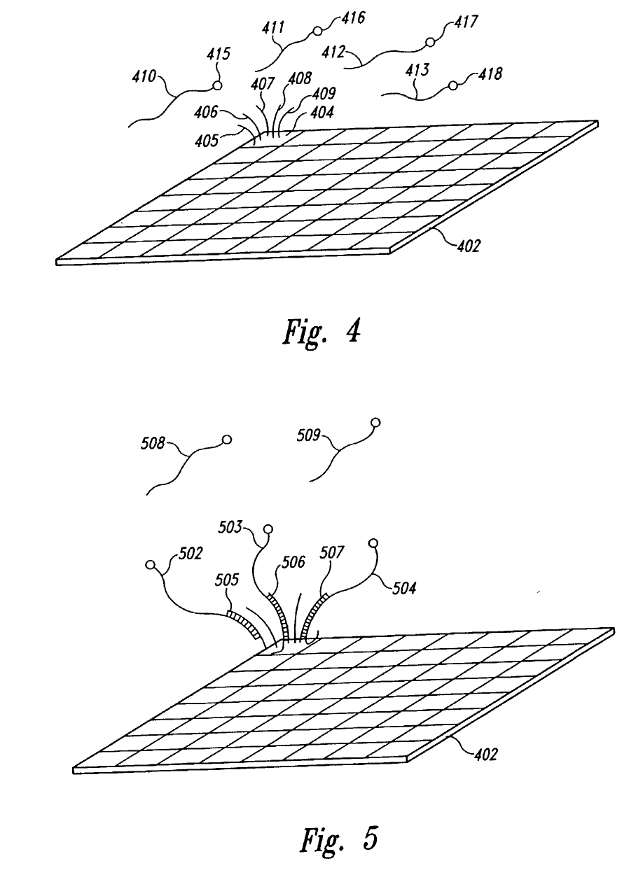

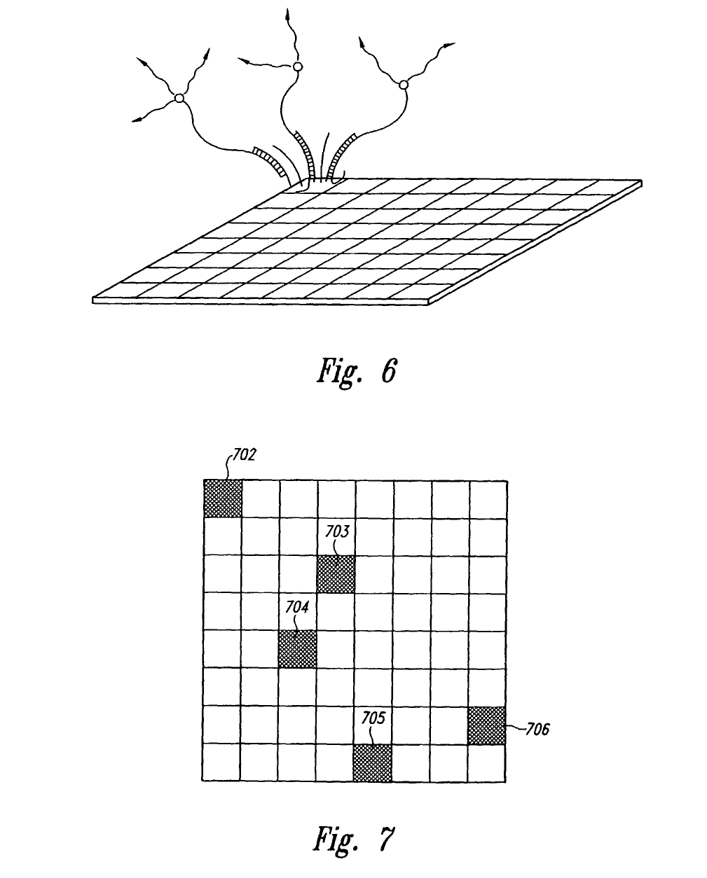

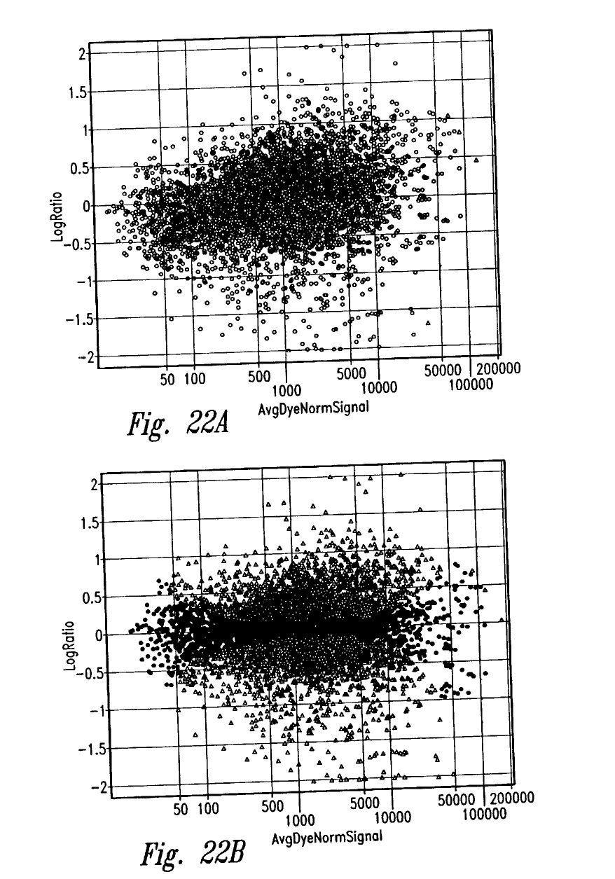

FIGS. 22A-B show plots of log expression ratios obtained from gene-expression differential comparison experiments with respect to net normalized feature-signal magnitudes. The log expression ratios are derived from globally normalized data sets, in FIG. 4A, and are derived from data sets renormalized by the method of the present invention in FIG. 4B. In both figures, feature-signal magnitudes of normalizing features are represented by circles, while feature-signal magnitudes of non-normalizing features are represented by triangles. Again, comparing FIG. 4A to 4B, it can be seen that the axis of symmetry for the distribution of log ratios in FIG. 4A is slightly tilted upward as a result of a systematic normalization error, while the approximate axis of symmetry of the distribution of log ratios in FIG. 4A is essential horizontal.

A First C++-Like Pseudocode Implementation of One Embodiment of the Present Invention

In this subsection, a C++-like pseudocode implementation of one embodiment of the present invention is provided. This first, basic embodiment demonstrates the rank-ordering-based normalizing feature selection and RTF construction. Detailed descriptions of function members and detailed implementations for ancillary classes not directly related to the present invention are not provided, in the interests of brevity. There are an almost limitless number of ways in which to implement the normalizing method of the present invention. The C++-like pseudocode implementation is provided for illustration, and is not intended to in any way limit the scope of the claimed invention.

The C++-like pseudocode employs a number of standard C libraries:

#include <stdlib.h>
#include <stdio.h>
#include <stdarg.h>
#include <math.h>
#include <float.h>

Next, a number of constant integer declarations and an enumeration, used in following function members, are provided:

1 const int MAX_COL = 100;
2 const int MAX_ROW = 100;
3 const int MAX_NUM = MAX_COL * MAX_ROW;
4 const double BIG_NUM = 4000000000;
5 const int halfWindow = 10;
6 const int winMul = 8;
7 const int xWin = 8;
8 enum rawMode {OLD, NEW, MODIFY};

The constants “MAX_COL” and “MAX_ROW,” declared above on lines 1-2 specify the maximum data-set size. Of course, in a commercial application, much larger data sets are processed, and correspondingly larger data structures need to be employed. A single molecular array may contain thousands to tens of thousands of features. Thus, certain of the 1-dimensional and 2-dimensional arrays, used to store feature-signal magnitudes and feature identifiers, need to of sufficient size to accommodate tens of thousands of individual feature-signal magnitudes and feature identifiers. The constant “MAX_NUM,” declared above on line three, is the maximum index for 1-dimensional arrays that contain feature-signal magnitude data sets or feature identifiers. The constant “BIG_NUM” is a large-integer placeholder to represent the feature-signal magnitudes of outlier features used during sorting of data sets. The constants “halfWindow,” “winMul,” and “xWin,” declared above on lines 5-7, are used to specify the size of the sliding window that traverses one data-set array during normalizing feature selection by the rank-ordering method described in the previous subsection. Finally, the enumeration “rawMode” is used to specify whether to use a data set stored in a file, construct a new simulated data set, or modify an existing data set while instantiating a instance of the class “rawdata,” to be described below.

Next, several type declarations are provided:

1 typedef struct rawfeatur
2 {

| | |
|---|---|
| 3 | bool outlier; |
| 4 | int val; |

5 } rawFeature;
6 typedef struct normfeatur
7 }

| | |
|---|---|
| 8 | bool outlier; |
| 9 | double val; |

10 } normFeature;

The type definitions “rawFeature” and “normFeature,” declared above on lines 1-10, store the feature-signal magnitude and outlier status for a single feature in a data set. The type “rawFeature” is used to store integer-based, raw-data-feature-signal magnitudes, while the type “normFeature” is used to store floating-point-based normalized feature-signal magnitudes and outlier status for features in normalized, or partially normalized, data sets.

Next, a declaration for a class, an instance of which stores a raw-data data set, is provided:

1 class rawdata
2 {

| | |
|---|---|
| 3 | private: |

| | |
|---|---|
| 4 | FILE *fl; |
| 5 | rawFeature data[MAX_COL][MAX_ROW]; |
| 6 | int maxCol; |
| 7 | int maxRow; |
| 8 | bool open; |
| 9 | bool storeData( ); |

| | |
|---|---|
| 10 | public: |

| | |
|---|---|
| 11 | int getFeature(int i, int j) |

| | |
|---|---|
| 12 | {if (i < maxRow && j < maxCol) return data[i][j].val; |
| 13 | else return |

| | |
|---|---|
| 14 | bool outlying(int i, int j) |

| | |
|---|---|
| 15 | {if (i < maxRow && j < maxCol) return data[i][j].outlier; |
| 16 | else return false;}; |

| | |
|---|---|
| 17 | int getMaxRow( ) {return maxRow;}; |
| 18 | int getMaxCol( ) {return maxCol;}; |
| 19 | rawdata(char* name, rawdata* rd, rawMode r, int maxrow, |

| | |
|---|---|
| 20 | int maxcol); |

| | |
|---|---|
| 21 | ˜rawdata( ); |

22 };

The class “rawdata” includes a 2-dimensional array member “data,” declared above on line 5, for storing a raw-data data set. The class “rawdata” includes the following function members: (1) “getFeature,” declared above on lines 11-3, which returns the feature-signal magnitude for a specified feature; (2) “outlying,” which returns the outlier status for a specified feature; (3) “getMaxRow,” which returns the number of rows in the 2-dimensional-array representation of the data set; (4) “getMaxCol,” declared above on line 18, which returns the number of columns in the 2-dimemisonal-array representation of the data set; and (5) a constructor and destructor declared on lines 19-21. Note that the C++-Iike pseudocode implementation generally employs simulated data that is constructed according to the arguments specified for the constructor, although an existing, file-based data set having a compatible format may also be used.

Next, a similar class “normadata” is declared for storing floating-point-based, normalized feature-signal magnitudes for normalized data sets:

1 class normdata
2 {

| | |
|---|---|
| 3 | private: |

| | |
|---|---|
| 4 | FILE *fl; |
| 5 | normFeature data[MAX_NUM]; |
| 6 | int maxCol; |
| 7 | int maxRow; |
| 8 | int maxNum; |
| 9 | bool open; |

| | |
|---|---|
| 10 | public: |

| | |
|---|---|
| 11 | double getFeature(int i, int j) |

| | |
|---|---|
| 12 | {int dex = (i * maxCol) + j; |
| 13 | if (dex < maxNum) return data[dex].val; |
| 14 | else return 0;}; |

| | |
|---|---|
| 15 | bool outlying(int i, int j) |

| | |
|---|---|
| 16 | {int dex = (i * maxCol) + j; |
| 17 | if (dex < maxNum) return data[dex].outlier; |
| 18 | else return false;}; |

| | |
|---|---|
| 19 | double getFeature(int i) |

| | |
|---|---|
| 20 | {if (i < maxNum) return data[i].val; |
| 21 | else return 0;}; |

| | |
|---|---|
| 22 | bool outlying(int i) |

| | |
|---|---|
| 23 | {if (i < maxNum) return data[i].outlier; |
| 24 | else return 0;}; |

| | |
|---|---|
| 25 | int getMaxNum( ) {return maxNum;}; |
| 26 | bool storeData( ); |
| 27 | void setFeature(double d, int i, int j) |

| | |
|---|---|
| 28 | {int dex = (i * maxCol) + j; |
| 29 | if (dex < maxNum) data[dex].val = d;}; |

| | |
|---|---|
| 30 | void setFeature(double d, int i) |

| | |
|---|---|
| 31 | {if (i < maxNum) data[i].val = d;}; |

| | |
|---|---|
| 32 | void setOutlier(int i) |

| | |
|---|---|
| 33 | {if (i < maxNum) data[i].outlier = true;}; |

| | |
|---|---|
| 34 | normdata(char* name, rawdata* rd); |
| 35 | ˜normdata( ); |

36 };

The class “normdata” is quite similar to the previously described class “rawdata,” with the exception that the normalized data in the class “normdata” is stored in a floating-point array, rather than in an integer array. The class “normdata” includes function members similar to those of the class “rawdata,” as well as the following, additional function members: (1) “getFeature,” declared above on line 19, which is an overloaded version of the function member “getFeature” declared on line 11, which accesses the normalized data set as a 1-dimensional array via a single specified index; (2) “getMaxNum,” declared above on line 25, which returns the number of feature-signal magnitudes in the data set contained in an instance of the class “normdata;” and (3) function members that store the normalized data set into a file and that set the feature-signal magnitude and outlier status for a specified feature, declared above on lines 27-33.

Next, the class “normalizer” is declared:

1 class normalizer
2 {
3 private:

| | |
|---|---|
| 4 | normdata* red; |
| 5 | normdata* green; |
| 6 | int numOK; |
| 7 | int funcNum; |
| 8 | bool OK; |
| 9 | int greenRanked[MAX_NUM]; |
| 10 | int redRanked[MAX_NUM]; |
| 11 | double normFuncY[MAX_NUM]; |
| 12 | double normFuncX[MAX_NUM]; |
| 13 | double avXinc; |
| 14 | void sort(int* l, int* r, normdata* nd); |
| 15 | double mid(int* i, int* j, int* k, normdata* nd); |
| 16 | void arithmeticMean( ); |
| 17 | void geometricMean( ); |
| 18 | void rank( ); |
| 19 | void smooth( ); |
| 20 | void norm( ); |

| | |
|---|---|
| 21 | public: |

| | |
|---|---|
| 22 | bool getOK( ) {return OK;}; |
| 23 | void normalize(bool arithmetic); |
| 24 | normalizer(normdata* r, normdata* g); |

25 };

The class “normalizer” includes the following data members: (1) “red” and “green,” declared above on lines 4-5, which reference two different data sets contained in instances of the class “normdata” that are to be normalized, referred to below as the “red and green data sets;” (2) “numOK,” declared above on line 6, which contains the number of features that are not outlying features in either or both the red and green data sets; (3) “funcNum,” declared above on line 7, which contains the number of data points in the RTF constructed from the red and green data sets, or, in other words, the number of normalizing features; (4) “OK,” a Boolean data member that indicates whether or not the input data, contained in the instances of the class “normData” referenced by data members “green” and “red,” has been successfully incorporated into an instance of the class “normalizer;” (5) “greenRanked” and “redRanked,” sorting arrays corresponding to the data sets referenced by data members “green” and “red”, respectively; (6) “normFuncY” and “normFuncX,” declared above on lines 11-12, that together compose an RTF array; and (7) “avXinc,” which contains the average X-axis difference between successive points in the RTF. The class “normalizer” includes the following private function members: (1) “sort,” which sorts indices stored in a sorting array based on the feature-signal magnitudes of the features corresponding to the indices; (2) “mid,” which returns the intermediate value of the values referenced by three integer pointers supplied as arguments; (3) “arithmeticMean,” declared above on line 16, which computes the arithmetic mean of the feature-signal magnitudes of the red and green data sets and globally normalizes the data set based on the computed arithmetic mean; (4) geometricMean,” which computes the geometric mean for the feature-signal magnitudes of the red and green data sets and globally normalizes the feature-signal magnitudes based on the geometric mean; (5) “rank,” which rank orders the non-outlying features of the red and green data sets, selects the normalizing features, and constructs the RTF; (6) “smooth,” which smoothes the constructed RTF; and (7) “norm,” which rescales the green data set to the red data set based on the smoothed RTF. The class “normalizer” includes the following public function members: (1) “getOK,” declared above on line 22, which returns the value of the data member “OK;” (2) “normalize,” which normalizes the initially unnormalized data in the red and green data sets, where the argument “arithmetic” specifies whether or not it is the arithmetic mean for global normalization, the alternative being the geometric mean; and (3) a constructor, declared above on line 24.

Implementations for certain of the function members for the class normalizer are next provided. First, implementations for the function members “mid” and “source” are provided, below:

1 double normalizer::mid(int* i, int* j, int* k, normdata* nd)
2 {

| | |
|---|---|
| 3 | if (nd->getFeature(*i) <= nd->getFeature(*j)) |
| 4 | { |

| | |
|---|---|
| 5 | if (nd->getFeature(*j) <= nd->getFeature(*k)) |

| | |
|---|---|
| 6 | return nd->getFeature(*j); |

| | |
|---|---|
| 7 | else return nd->getFeature(*k); |

| | |
|---|---|
| 8 | } |

9 else

| | |
|---|---|
| 10 | { |

| | |
|---|---|
| 11 | if (nd->getFeature(*i) <= nd->getFeature(*k)) |

| | |
|---|---|
| 12 | return nd->getFeature(*i); |

| | |
|---|---|
| 13 | else if (nd->getFeature(*k) > nd->getFeature(*j)) |

| | |
|---|---|
| 14 | return nd->getFeature(*k); |

| | |
|---|---|
| 15 | else return nd->getFeature(*j); |

| | |
|---|---|
| 16 | } |

17 }
1 void normalizer::sort(int* l, int* r, normdata* nd)
2 {

| | |
|---|---|
| 3 | int t, numP = r −l + 1; |
| 4 | double pivot; |
| 5 | int*m, *i, *j; |
| 6 | if (numP <= 1) return; |
| 7 | if (numP == 2) |
| 8 | { |

| | |
|---|---|
| 9 | if (nd->getFeature(*r) >= nd->getFeature(*l)) return; |
| 10 | else |
| 11 | { |

| | |
|---|---|
| 12 | t = *r; |
| 13 | *r = *l; |
| 14 | *l = t; |
| 15 | return; |

| | |
|---|---|
| 16 | } |

| | |
|---|---|
| 17 | } |
| 18 | t = numP / 2; |
| 19 | m = l + t; |
| 20 | pivot = mid(l, m, r, nd); |
| 21 | i =l; |
| 22 | j = r; |
| 23 | while (true) |
| 24 | { |

| | |
|---|---|
| 25 | while (i < j && nd->getFeature(*i) < pivot) i++; |
| 26 | while (j > i && nd->getFeature(*j) > pivot) j−−; |
| 27 | if (i == j) |
| 28 | { |

| | |
|---|---|
| 29 | if (nd->getFeature(*i) <= pivot) |
| 30 | { |

| | |
|---|---|
| 31 | sort(l, i, nd); |
| 32 | sort(j + 1, r, nd); |

| | |
|---|---|
| 33 | } |
| 34 | else |
| 35 | { |

| | |
|---|---|
| 36 | sort(l, i − 1, nd); |
| 37 | sort(j, r, nd); |

| | |
|---|---|
| 38 | } |
| 39 | break; |

| | |
|---|---|
| 40 | } |
| 41 | else if (i < j) |
| 42 | { |

| | |
|---|---|
| 43 | t = *i; |
| 44 | *i = *j; |
| 45 | *j = t; |

| | |
|---|---|
| 46 | } |
| 47 | if (i == j − 1) |
| 48 | { |

| | |
|---|---|
| 49 | sort(l, i, nd); |
| 50 | sort(j, r, nd); |
| 51 | break; |

| | |
|---|---|
| 52 | } |
| 53 | i++; |
| 54 | j−; |

| | |
|---|---|
| 55 | } |

56 }

The normalizer function members “mid” and “sort,” shown above, implement a modified quicksort procedure for sorting a sorting array based on the globally normalized feature-signal magnitudes indexed by the indices in the sorting array. The arguments to the function member “sort” include pointers to the left-most and right-most elements of the 1-dimensional sorting array, and a pointer to an instance of the class “normdata” containing the normalized data set with which the sorting array is associated. Both of the above function members are straightforward, and are not therefore further described in the interest of brevity.

Next, an implementation for the function member “arithmeticMean” is provided:

1 void normalizer::arithmeticMean( )
2 {

| | |
|---|---|
| 3 | int i, j; |
| 4 | double gAv = 0, rAv = 0; |
| 5 | numOK = 0; |
| 6 | for (i = 0; i < red->getMaxNum( ); i++) |
| 7 | { |

| | |
|---|---|
| 8 | if (red->outlying(i)) |
| 9 | { |

| | |
|---|---|
| 10 | green->setOutlier(i); |
| 11 | red->setFeature(BIG_NUM, i); |
| 12 | green->setFeature(BIG_NUM, i); |

| | |
|---|---|
| 13 | } |
| 14 | else if (green->outlying(i)) |
| 15 | { |

| | |
|---|---|
| 16 | red->setOutlier(i); |
| 17 | green->setFeature(BIG_NUM, i); |
| 18 | red->setFeature(BIG_NUM, i); |

| | |
|---|---|
| 19 | } |
| 20 | else |
| 21 | { |

| | |
|---|---|
| 22 | rAv += red->getFeature(i); |
| 23 | gAv += green->getFeature(i); |
| 24 | numOK++; |

| | |
|---|---|
| 25 | } |

| | |
|---|---|
| 26 | } |
| 27 | rAv = rAv / numOK; |
| 28 | gAv = gAv / numOK; |
| 29 | for (i = 0; i < red->getMaxNum( ); i++) |
| 30 | { |

| | |
|---|---|
| 31 | if (!red->outlying(i)) |
| 32 | { |

| | |
|---|---|
| 33 | red->setFeature(red->getFeature(i) / rAv, i); |
| 34 | green->setFeature(green->getFeature(i) / gAv, i); |

| | |
|---|---|
| 35 | } |

| | |
|---|---|
| 36 | } |

37 }

The function member “arithmeticMean,” provided above, calculates the arithmetic mean for non-outlying feature-signal magnitudes for both the red and green data sets, and then globally normalizes both the red and green data sets, using the calculated arithmetic means. In the for-loop of lines 6-26, function member “arithmeticMean” traverses all the features in both the red and green data sets. Each feature that is not an outlier in both the red and green data sets is used in summing the feature-signal magnitudes for the red and green data sets, on lines 22-23. Local variables “rAv” and “gAv” contain the summed feature-signal magnitudes, and data member “numOK” contains the number of non-outlying features processed. For features that are outliers in either or both of the red and green data sets, the feature-signal magnitudes are set to the constant value “BIG_NUM,” on lines 11-12 and 17-18, to indicate that the features are outliers, and to facilitate subequent sorting. The red and green data-set arithmetic means are then calculated, on lines 27-28, by dividing the accumulated sum of the feature-signal magnitudes for both data sets by the number of valid, or non-outlying, features stored in the data member “numOK.” Finally, in the for-loop of lines 29-36, both the red and green data sets are globally normalized by setting the feature-signal magnitude for each signal to be the initial feature-signal magnitude divided by the arithmetic mean for the data set.

The following function member “geometric mean” calculates the geometric mean, rather than the arithmetic mean, for both the red and green data sets, and globally normalizes both data sets based on the calculated geometric means. The implementation of the function member “geometric mean” is quite similar to that of the function member “arithmetic mean” described above, and will therefore not be further discussed:

1 void normalizer::geometricMean( )
2 {

| | |
|---|---|
| 3 | int i; |
| 4 | double gAv = 0, rAv = 0; |
| 5 | numOK = 0; |
| 6 | for (i = 0; 1 < red->getMaxNum( ); i++) |
| 7 | { |

| | |
|---|---|
| 8 | if (red->outlying(i)) |
| 9 | { |

| | |
|---|---|
| 10 | green->setOutlier(i); |
| 11 | red->setFeature(BIG_NUM, i); |
| 12 | green->setFeature(BIG_NUM, i); |

| | |
|---|---|
| 13 | } |
| 14 | else if (green->outlying(i)) |
| 15 | { |

| | |
|---|---|
| 16 | red->setOutlier(i); |
| 17 | green->setFeature(BIG_NUM, i); |
| 18 | red->setFeature(BIG_NUM, i); |

| | |
|---|---|
| 19 | } |
| 20 | else |
| 21 | { |

| | |
|---|---|
| 22 | rAv += log10(red->getFeature(i)); |
| 23 | gAv += log 10(green->getFeature(i)); |
| 24 | numOK++; |

| | |
|---|---|
| 25 | } |

| | |
|---|---|
| 26 | } |
| 27 | rAv = pow(10, rAv / numOK); |
| 28 | gAv = pow(10, gAv / numOK); |
| 29 | for (i = 0; i < red->getMaxNum( ); i++) |
| 30 | { |

| | |
|---|---|
| 31 | if (!red->outlying(i)) |
| 32 | { |

| | |
|---|---|
| 33 | red->setFeature(red->getFeature(i) / rAv, i); |
| 34 | green->setFeature(green->getFeature(i) / gAv, i); |

| | |
|---|---|
| 35 | } |

| | |
|---|---|
| 36 | } |

37 }

Next, an implementation for the function member “rank” is provided, below:

1 void normalizer: :rank( )
2 {

| | |
|---|---|
| 3 | int i, j, k, m; |
| 4 | int nxtG, nxtR; |
| 5 | double nxtX; |
| 6 | avXinc = 0; |
| 7 | for (i = 0; i < red->getMaxNum( ); i++) |
| 8 | { |

| | |
|---|---|
| 9 | greenRanked[i] = i; |
| 10 | redRanked[i] = i; |

| | |
|---|---|
| 11 | } |
| 12 | sort(&(greenRanked[0]), &(greenRanked[green->getMaxNum( ) - 1]), green); |
| 13 | sort(&(redRanked[0]), &(redRanked[red->getMaxNum( ) − 1]), red); |
| 14 | funcNum = 0; |
| 15 | for (i = −halfWindow, j = 0, k = j + halfWindow; j < numOK; i++, j++, k++) |
| 16 | { |

| | |
|---|---|
| 17 | nxtG = greenRanked[j]; |
| 18 | if (i < 0) i = 0; |
| 19 | if (k > numOK) k = numOK; |
| 20 | for (m = i; m < k; m++) |
| 21 | { |

| | |
|---|---|
| 22 | if (nxtG == redRanked[m]) |
| 23 | { |

| | |
|---|---|
| 24 | nxtR = redRanked[m]; |
| 25 | nxtX = green->getFeature(nxtG); |
| 26 | normFuncY[funcNum] = red->getFeature(nxtR) / nxtX; |
| 27 | if (funcNum > 0) avXinc += |

| | |
|---|---|
| 28 | nxtX − normFuncX[funcNum − 1]; |

| | |
|---|---|
| 29 | normFuncX[funcNum++] = nxtX; |
| 30 | break; |

| | |
|---|---|
| 31 | } |

| | |
|---|---|
| 32 | } |

| | |
|---|---|
| 33 | } |
| 34 | avXinc = avXinc / (funcNum − 1); |

35 }

The function member “rank” carries out selection of the normalizing features, as described in the previous subsection. First, in the for-loop of line 7-11, the sorting arrays “greenRank” and “redRank” are initialized to contain a monotomically increasing set of 1-dimensional array indices of features in the green and red data sets. Then, on lines 12-13, the function member “sort” is called twice to sort each sorting array based on the feature-signal magnitudes of the features in the corresponding globally normalized data sets referenced by data members “green” and “red.” Finally, in the for-loop of lines 13-33, a green-sorting-array element pointer j traverses the sorting array associated with the green data set, and a sliding window defined by left and right element pointers i and k traverses the red sorting array, as described in the previous subsection, to select features that are closely ranked together in both sorting arrays, or, in other words, have similar rank orders in both the red and green data sets. Note that the size of the sliding window is determined, arbitrarily, by the constant integer “halfWindow.” In alternative embodiments, the size of the sliding window may be varied, or may be depend on data-related factors. For example, the sliding window may be determined both as a number of sorting-array elements, and then widened or narrowed to encompass a desired range of feature-signal magnitudes. The outer for-loop of lines 15-33 increments the sliding window left and right element pointers i and k and element pointer j with each iteration to traverse the sorting arrays. The inner for-loop of lines 20-32 searches the elements currently covered by the sliding window for an element containing an index matching the index stored in the element pointed to by the element pointer, using the innerfor-loop element pointer m. If an index is found within an element, referenced by element pointer m, covered by the sliding window equal to the index in the element referred to by the element pointer j, then the pair of indices in elements pointed to by element pointers m and j are used to fetch normalized red and green feature-signal magnitudes to construct an RTF data point. The y-value of the data point is stored in the array “normFuncY” on line 26, and the x-value for the data point is stored in the array “normFuncX” on line 29. The data member “avXinc” is incremented with each addition of a data point, so that, on line 34, the average x-axis increment can be calculated and stored into data member “avXinc.”

Next, an implementation for the function member “smooth” is provided:

1 void normalizer::smooth( )
2 {

| | |
|---|---|
| 3 | int i, j, k, l; |
| 4 | double halfWindow = avXinc * winMul; |
| 5 | double low, high, res; |
| 6 | for (i = 0; i < funcNum; i++) |
| 7 | { |

| | |
|---|---|
| 8 | low = normFuncY[i] − halfWindow; |
| 9 | high = normFuncY[i] + halfWindow; |
| 10 | for (j = i − 1; j >= 0 && i − j <= x Win;j−−) |
| 11 | { |

| | |
|---|---|
| 12 | if (normFuncY[j] <= low) break; |

| | |
|---|---|
| 13 | } |
| 14 | j = j + 1; |
| 15 | for (k = i + 1; k < funcNum && k − i < x Win; k++) |
| 16 | { |

| | |
|---|---|
| 17 | if (normFuncY[k] >= high) break; |

| | |
|---|---|
| 18 | } |
| 19 | if (k − j < 2) continue; |
| 20 | res = 0; |
| 21 | for (l = j; l < k; l++) |
| 22 | { |

| | |
|---|---|
| 23 | res += normFuncY[1]; |

| | |
|---|---|
| 24 | } |
| 25 | normFuncY[i] = res / (k − j); |

| | |
|---|---|
| 26 | } |

27 }

The function member “smooth” carries out a simple local average smoothing of the RTF. In the for-loop of lines 6-26, a sliding window traverses the RTF arrays “normFuncY” and “normFuncX,” replacing the central y-value of the sliding window with the average of the y-values of all elements covered by the sliding window. This is a very simple, if inelegant, smoothing process, but, in combination with rank-order-based normalizing feature selection, is adequate to provide reasonable green to red data set resealing.

Next, an implementation of the function member “norm” is provided:

1 void normalizer::norm( )
2 {

| | |
|---|---|
| 3 | int i, j, nxt; |
| 4 | double xRatio, yValue; |
| 5 | j = 0; |
| 6 | for (i = 0; i < numOK; i++) |
| 7 | { |

| | |
|---|---|
| 8 | nxt = greenRanked[i]; |
| 9 | while (normFuncX[j] < green->getFeature(nxt) && j < numOK) j++; |
| 10 | if (j == 0 \|\| j == numOK) continue; |
| 11 | if (normFuncX[j] == green->getFeature(nxt)) |
| 12 | { |

| | |
|---|---|
| 13 | green->setFeature(green->getFeature(nxt) * |

| | |
|---|---|
| 14 | normFuncY[j], nxt); |

| | |
|---|---|
| 15 | } |

| | |
|---|---|
| 16 | else |
| 17 | { |

| | |
|---|---|
| 18 | xRatio = (green->getFeature(nxt) − normFuncX[j − 1]) / |

| | |
|---|---|
| 19 | (normFuncX[j] − normFuncX[j−1]); |

| | |
|---|---|
| 20 | yValue = (normFuncY[j] − normFuncY[j−1]) * xRatio; |
| 21 | green->setFeature(green->getFeature(nxt)* |

| | |
|---|---|
| 22 | (normFuncY[j] + yValue), nxt); |

| | |
|---|---|
| 23 | } |

| | |
|---|---|
| 24 | } |

25 }

The function member “norm” determines an RTF-based red-to-green ratio for each globally normalized green feature-signal magnitude, and then multiplies the red-to-green ratio by the corresponding green feature-signal magnitude in order to produce a rescaled green feature-signal magnitude. Thus, function member “norm” interpolates red-to-green ratios from the smoothed RTF and applies the red-to-green ratio so obtained in order to rescale the green feature-signal magnitudes to the red data set. In the for-loop of lines 6-24, the next green-feature index to rescale is obtained, on line 8, from a currently considered element in the green-data-set sorting array. Then, the smoothed RTF is searched, on line 9, for an RTF data point with x-value just greater than the green feature-signal magnitude of the next green feature. If an exact match is found in the smoothed RTF, on line 11, then the corresponding red-to-green data ratio is used to rescale the next green feature on lines 13-14. Otherwise, a red-to-green ratio is interpolated, on lines 18-20, and is then used to rescale the green feature on lines 21-22.

Next, an implementation for the function member “normalize” is provided:

1 void normalizer::normalize(bool arithmetic)
2 {

| | |
|---|---|
| 3 | if (OK) |
| 4 | { |

| | |
|---|---|
| 5 | if (arithmetic) arithmeticMean( ); |
| 6 | else geometricMean( ); |
| 7 | rank( ); |
| 8 | smooth( ); |
| 9 | norm( ); |

| | |
|---|---|
| 10 | } |

11 }

The function member “normalize” carries out the steps of the described embodiment of the present invention. First, either global normalization using an arithmetic mean or global normalization using a geometric mean is carried out on line 5 or line 6, depending on the value of the argument “arithmetic.” Then, on line 7, normalizing features are selected by the rank-ordering method, and an initial RTF is created. Next, on line 8, the initial RTF is smoothed. Finally, on line 9, the green data set is rescaled to the red data set using the smoothed RTF created on lines 7 and 8.

A constructor for the class “normalizer” is next provided:

1 normalizer::normalizer(normdata* r, normdata* g)
2 {

| | |
|---|---|
| 3 | if (r->getMaxNum( ) != g->getMaxNum( )) |
| 4 | { |

| | |
|---|---|
| 5 | OK = false; |
| 6 | return; |

| | |
|---|---|
| 7 | } |
| 8 | red = r; |
| 9 | green = g; |

10 }

Finally, a simple main routine is provided, to illustrate the use of instances of the classes “rawdata,” “normdata,” and “normalizer:”

1 const int Mrow = 50;
2 const int Mcol = 50;
3 const int Mnum = Mrow * Mcol;
4 int main(int argc, char* argv[])
5 {

| | |
|---|---|
| 6 | char inRName[100] = “C:\\inputRedData”; |
| 7 | char inGName[100] = “C:\\inputGreenData”; |
| 8 | char outRName[100] = “C:\\outputRedData”; |
| 9 | char outGName[100] = “C:\\outputGreenData”; |
| 10 | rawdata inRD(inRName, (rawdata*)NULL, OLD, Mrow, Mcol); |
| 11 | rawdata inGD(inGName, &inRD, OLD, Mrow, Mcol); |
| 12 | normdata outDG(outGName, &inGD); |
| 13 | normdata outDR(outRName, &inRD); |
| 14 | normalizer Norm(&outDR, &outDG); |
| 15 | Norm.normalize(false); |
| 16 | return 0; |

17 }

The pseudocode implementation simulates the raw data. The size of the simulated data sets is specified by the constant integers “Mrow,” “Mcol” and “Mnum,” declared above on lines 1-3. Instances of the classes “rawdata” and “normdata” are declared for the input and output of red and green data sets on lines 10-13. Note that, in declaring an instance of the class “normdata,” an instance of the class “rawdata” is provided as an argument. The constructor for the class “normdata” inputs the raw data into the floating point, 2-dimensional array within the instance of the class “normdata.” Next, on line 14, an instance of the class “normalizer” is declared, with references to the red and green data sets provided as arguments. Finally, on line 15, the function member “normalize” is invoked to carry out global normalization of the red and green data sets and green-to-red data-set resealing, according to the described embodiment of the present invention.

A Second, Improved C++-Like Pseudocode Implementation of One Embodiment of the Present Invention

Although the first embodiment of the method of the present invention, described in the previous subsection, can be used to satisfactorily normalize molecular array data sets, additional features described in the current subsection can provide a more desirable normalization for certain types of data. One feature involves distributing corrections fairly between the \overline{C1} and \overline{C2} data sets during the normalization process, rather than resealing the \overline{C2} data set to the \overline{C1} data set. Another feature involves using the LOWESS non-parametric approach to smoothing the RTF, and for computing the RTF value corresponding to a feature-signal magnitude that is used to correct globally normalized data sets \overline{C1} and \overline{C2}.

LOWESS is a curve-fitting method. In some curve-fitting techniques, an underlying mathematical model is assumed, and a curve conforming to the mathematical model is brought into conformance with a set of data points by one of many different possible methods, including minimizing differences between the positions of the data points relative to the curve. By contrast. LOWESS does not assume a mathematical model, but instead smoothes a set of data points by following a trend in the data points.

LOWESS is a bivariate smoother for drawing a smooth curve through a scatter diagram. LOWESS finds a curve that is smooth, and that locally minimizes the variance of residuals, or prediction errors. LOWESS finds a smoothed fitted value, ŷᵢ, for each xᵢ by fitting a weighted regression to the points in the neighborhood of xᵢ. The points closest to xᵢ receive the greatest weight via a locally weighted regression part of the LOWESS method, and the weights are called “neighborhood weights.” LOWESS is an iterative method. Once the fitted values, ŷᵢ, have been found, residuals, rᵢ=yᵢ−ŷᵢ are used to determine a new set of weights, called “robustness weights,” so that points which have large residuals are down-weighted, and the locally weighted regression is repeated.

In practice, LOWESS depends on the choice of a smoothing parameter, ƒ, 0<ƒ<=1, the fraction of the data points to be considered in the calculation of ŷᵢ. Choosing ƒ=0.5 means that only the r=[0.5 n] points closest to xᵢ have non-zero weights. Increasing the value of ƒ makes the fitted curve smoother, decreasing ƒ lets the curve follow the data more closely. Additional details about the LOWESS method can be found in widely available textbooks on statistics, statistical learning, and curve fitting techniques.

In the currently described embodiment of the present invention, a standard, publicly available LOWESS implementation is modified for application to the RTF smoothing and RTF value estimation described in previous subsections. A routine “lowess_correct” that represents a modification of the standard routine “lowess” is defined with the prototype:

lowess_correct (double *x, double *y, int n, double fitx, double f, double& ys, double *rw, Rboolean& ok)

The routine “lowess-correct( )” takes a LOWESS curve defined by a set of set of n normalization points, where point i is defined by (x[i], y[i]). The routine “lowess_correct( )” sorts the points by ascending values of x, calculates an array of weights for these points, rw[], and calculates the value of y along the curve, stored in ys, for any new input value x passed in as ƒitx. The routine “lowess_correct( )” accomplishes this by selecting a subset of ƒ*n consecutive normalization points, where 0<=ƒ<=1, such that fitx−min(norm_x)<=max(norm_x)−fitx where min(norm_x) and max(norm_x) are the minimum and maximum x values in the ƒ*n subset. The x and y values of this subset, along with the weights corresponding to this subset and ƒitx, are then passed to lowest( ), which returns the y value, ys, of the LOWESS curve at point ƒitx.

The routine “lowess_correct( )” differs from the standard routine “lowess( )” in that lowess( ) assumes that the normalization points are the same as the points to be corrected. Therefore, lowess( ) repeats the above steps for every i, setting fitx=×[i]. In addition, lowess( ) calculates weights based on the residuals between the LOWESS curve and the actual y values of the normalization points. The standard routine “lowess( )” estimates the curve iteratively, using these weights in calls to the standard routine “lowest( )” for subsequent iterations, and thereby refining the weights. The routine “lowess_correct( )” avoids these steps, since the weights on the normalization points have already been refined in an earlier call to lowess( ).

The distribution of RTF corrections fairly between the \overline{C1} and \overline{C2} data sets during the normalization process can be straightforwardly described in mathematical terms. The combined feature-signal magnitude for a feature can be described as:

\overline{s}ᵢ=log₁₀(\overline{s}ᵢ_{_{C1}}×ᵢ_{_{C2}})/2

The dye bias for a feature directed to target molecules having equal concentrations in both sample solution, referred to as an “unregulated feature” in gene-expression experiments, can be expressed as:

cᵢ=log₁₀(\overline{s}ᵢ_{_{C1}}/\overline{s}ᵢ_{_{C2}})

or, alternatively, as:

cᵢ=log₁₀(\overline{s}ᵢ_{_{C1}})−log₁₀(\overline{s}ᵢ_{_{C2}})

where cᵢ can be estimated from the RTF for the data sets by a LOWESS estimation method. The correction term cᵢ can be applied to the feature-signal magnitudes of both data sets for feature i, so that:

0=[log₁₀(\overline{s}ᵢ_{_{C1}})−(cᵢ/2)]−[log₁₀(\overline{s}ᵢ_{_{C2}})+(cᵢ/2)]

The corrected color-channel feature-signal magnitudes are then given as:

log₁₀(\tilde{s}ᵢ_{_{C1}})=log₁₀(\overline{s}ᵢ_{_{C1}})−(cᵢ/2)

log₁₀(\tilde{s}hd i_{_{C2}})=log₁₀(\overline{s}ᵢ_{_{C2}})+(Cᵢ/2)

The log ratio of the corrected color-channel feature-signal magnitudes for an unregulated feature is 0:

log₁₀(\tilde{s}ᵢ_{_{C1}}/\tilde{s}ᵢ_{_{C1}})=log₁₀(\tilde{s}ᵢ_{_{C1}})−log₁₀(\tilde{s}ᵢ_{_{C2}})

log₁₀(\tilde{s}ᵢ_{_{C1}})−log₁₀(\tilde{s}ᵢ_{_{C2}})=[log₁₀(\overline{s}ᵢ_{_{C1}})−(cᵢ/2)]−[log₁₀(\overline{s}ᵢ_{_{C2}})+(cᵢ/2)]=0

while the combined feature-signal magnitude is unchanged by the correction:

$$\begin{matrix}{log}_{10} ( {\tilde{s}}_{{i}_{C1}} \times {\tilde{s}}_{{i}_{C2}} ) / 2 = [ {log}_{10} ( {\tilde{s}}_{{i}_{C1}} ) + {log}_{10} ( {\tilde{s}}_{{i}_{C2}} ) ] / 2 \\ = [ [ {log}_{10} ( {\overline{s}}_{{i}_{C1}} ) - ( {c}_{i} / 2 ) ] + [ {log}_{10} ( {\overline{s}}_{{i}_{C2}} ) + ( {c}_{i} / 2 ) ] ] / 2 \\ = [ {log}_{10} ( {\overline{s}}_{{i}_{C1}} ) + {log}_{10} ( {\overline{s}}_{{i}_{C2}} ) ] / 2 \\ = {log}_{10} ( {\overline{s}}_{{i}_{C1}} \times {\overline{s}}_{{i}_{C2}} ) / 2\end{matrix}$$

Thus, in the second embodiment of a method of the present invention, rather than rescaling the \overline{C2} data set to the \overline{C1} data set, both data sets are corrected, by equally applying LOWESS-determined corrections to the feature-signal magnitudes of each feature, without altering the overall, combined feature-signal magnitude for the feature.

C++-like pseudocode implementations for those classes and function members of the first embodiment, described above in the previous subsection, that need to be altered to produce a C++-like pseudocode implementation of the currently described, second embodiment, are provided below. Only significant changes between the altered classes and function members and the corresponding classes and function members in the first embodiment are described in the accompanying text, in the interest of brevity.

In the current embodiment, additional data members are added to the class “normalizer” and function members have been altered, as follows:

1 class normalizer
2 {

| | |
|---|---|
| 3 | private: |

| | |
|---|---|
| 4 | normdata* red; |
| 5 | normdata* green; |
| 6 | int numOK; |
| 7 | bool OK; |
| 8 | int normFeatNum; |
| 9 | int greenRanked[MAX_NUM]; |
| 10 | int redRanked[MAX_NUM]; |
| 11 | double normFuncY[MAX_NUM]; |
| 12 | double normFuncX[MAX_NUM]; |
| 13 | double avXinc; |
| 14 | int normFeat[MAX_NUM]; |
| 15 | double normFuncLR[MAX_NUM]; |
| 16 | double normWeights[MAX_NUM]; |
| 17 | double normResiduals[MAX_NUM]; |
| 18 | double normX[MAX_NUM]; |
| 19 | double normY[MAX_NUM]; |
| 20 | int normRank[MAX_NUM]; |
| 21 | double RankedNormX[MAX_NUM]; |
| 22 | double RankedNormY[MAX_NUM]; |
| 23 | void sort(int* l, int* r, double* nd); |
| 24 | double mid(int* i, int* j, int* k, double* nd); |
| 25 | void sort(int* l, int* r, normdata* nd); |
| 26 | double mid(int* i, int* j, int* k, normdata* nd); |
| 27 | void arithmeticMean( ); |
| 28 | void geometricMean( ); |
| 29 | void rankfilter(double thresh); |
| 30 | void LOWESS_smooth(double f, int nsteps, double delta); |
| 31 | void LOWESS_norm(double f); |

| | |
|---|---|
| 32 | public: |

| | |
|---|---|
| 33 | bool getOK( ) {return OK;}; |
| 34 | void normalize(bool arithmetic); |
| 35 | normalizer(normdata* r, normdata* g); |

36 };

The class “normalizer” includes the following new data members: (1) “normFeat” and “normFeatNum,” the normalizing features and the number of normalizing features, respectively; (2) “normFuncLR,” an array member containing the smoothed RTF y-values; (3) “normWeights,” an array member containing the LOWESS weights resulting from the LOWESS smoothing of the RTF; (4) “normResiduals,” an array member containing the LOWESS residual values computed after a number of iterations of LOWESS smoothing of the RTF; (5) “normX” and “normY,” member arrays that together compose a stored RTF for a combined red and green feature-signal magnitude; and (6) “RankedNormX” and “RankedNormY,” member arrays that together compose a stored RTF for a combined red and green feature-signal magnitude sorted to be monotonically increasing in combined feature-signal magnitude. The following function members are substituted for similarly named function members in the first embodiment: (1) “rankfilter,” a rank-order-based normalizing feature selection function member; (2) “LOWESS_smooth,” a LOWESS-based RTF smoother; and (3) “LOWESS_norm,” a function member that uses a modified lowess_correct routine to estimate RTF y-values for combined red and green feature-signal magnitudes and normalize the red and green data sets based on the estimated RTF y-values.

The following two functions have the form of standard LOWESS functions, but are modified, as described above at the beginning of the current subsection:

1 void lowess(double *x, double *y, int n,

| | |
|---|---|
| 2 | double f, int nsteps, double delta, |
| 3 | double *ys, double *rw, double *res); |

1 Rboolean lowess_correct(double *x, double *y, int n, double fitx,

| | |
|---|---|
| 2 | double f, double &ys, double *rw); |

The function “lowess” creates a smooth curve for a set of data points. The function “lowess” receives the following six input and three output arguments: (1) “x,” the x-values for the data points to which a curve is to be fit; (2) “y,” the y-values for the data points to which a curve is to be fit; (3) “n,” the number of data points; (4) “f,” the window over which linear regression is carried out for each data point, with respect to the x-axis; (5) “nsteps,” the x-axis interval for the LOWESS smoothing method; (6) “delta,” a parameter that is always set to 0, in the current implementation; (7) “ys,” the estimated, or smoothed, y-values resulting from the LOWESS smoothing; (8) “rw,” the LOWESS weights associated with the estimated, or smoothed, y-values; and (9) the LOWESS residuals. The function “lowess_correct” computes the estimated y-value for a supplied x-value according to the smooth curve created by a previous call to the above-described function “lowess.” The function “lowess_correct” receives the following input and output arguments: (1) “x,” “y,” and “n,” identical to the identically named, first three arguments to the function “lowess;” (4) “fitx,” the x-value for which an estimated y-value is sought; and (5) “f,” identical to the identically named argument to the function “lowess;” (6) “ys,” the estimated y-value produced by the function “lowess_correct;” and (7) “rw,” the LOWESS weights determined by a previous call to the function “lowess.”

An overloaded “sort” function member has been altered to take, as the final argument, a pointer to the first element of a 1-dimensional array that contains globally normalized, combined feature-signal magnitudes:

1 void normalizer::sort(int* 1, int* r, double* nd);

The following for-loop is added, in the currently described embodiment, to the end of the arithmeticMean and geomerticMean function members, in order to convert globally normalized feature-signal magnitudes to units of milliNorms:

1 for (i = 0; i < red->getMaxNum( ); i++)
2 {

| | |
|---|---|
| 3 | red->setFeature(red->getFeature(i) * 1000 / rAv, i); |

4 green->setFeature(green->getFeature(i) * 1000 / gAv, i);
5 }

In the current embodiment, a different rank-ordering-based normalizing feature selection method is employed, as follows:

1 void normalizer::rankfilter(double thresh)
2 {

| | |
|---|---|
| 3 | int i; |
| 4 | int greenRanks[MAX_NUM]; |
| 5 | int redRanks[MAX_NUM]; |
| 6 | avXinc = 0; |
| 7 | for (i = 0; i < red->getMaxNum( ); i++) |
| 8 | { |

| | |
|---|---|
| 9 | greenRanked[i] = i; |
| 10 | redRanked[i] = i; |

| | |
|---|---|
| 11 | } |
| 12 | sort(&(greenRanked[0]), &(greenRanked[green->getMaxNum( ) − 1]), green); |
| 13 | sort(&(redRanked[0]), &(redRanked[red->getMaxNum( ) − 1]), red); |
| 14 | for (i = 0; i < red->getMaxNum( ); i++) |
| 15 | { |

| | |
|---|---|
| 16 | greenRanks[greenRanked[i]] = i; |
| 17 | redRanks[redRanked[i]] = i; |

| | |
|---|---|
| 18 | } |
| 19 | normFeatNum = 0; |
| 20 | for (i = 0; 1 < red->getMaxNum( ); i++) |
| 21 | { |

| | |
|---|---|
| 22 | if (fabs(redRanks[i] − greenRanks[i])/red->getMaxNum( ) <= thresh) |
| 23 | { |

| | |
|---|---|
| 24 | normFeat[normFeatNum++] = i; |

| | |
|---|---|
| 25 | } |

| | |
|---|---|
| 26 | } |

27 }

In the function member “rankfilter,” above, after the green and red data sets are first sorted, by feature-signal magnitude, local arrays “greenRanks” and “redranks” are initialized, in the for-loop of lines 14-18, to contain the positions of features in the sorting arrays. Thus, for example, redRanks[i] contains the position of feature i in the red sorting array redRanked. Then, in the for-loop of lines 20-26, features for which the absolute difference in corresponding positions, or ranks, in the two sorting arrays are less than the threshold value “thresh” are selected as normalizing features.

In the following new smoothing function member, the LOWESS method is employed to fit a curve to the RTF data points corresponding to the normalizing features:

1 void normalizer::LOWESS_smooth(double f, int nsteps, double delta)
2 {

| | |
|---|---|
| 3 | int i; |
| 4 | for (i = 0; i < normFeatNum; i++) |
| 5 | { |

| | |
|---|---|
| 6 | normX[i] =log10(red->getFeature(normFeat[i])* |

| | |
|---|---|
| 7 | green->getFeature(normFeat[i]))/2.0; |

| | |
|---|---|
| 8 | normY[i] = log10(red->getFeature(normFeat[i])/ |

| | |
|---|---|
| 9 | green->getFeature(normFeat[i])); |

| | |
|---|---|
| 10 | normRank[i] = i; |

| | |
|---|---|
| 11 | } |
| 12 | sort(&(normRank[0]), &(normRank[normFeatNum − 1]), &(normX[0])); |
| 13 | for (i = 0; i < normFeatNum; i++) |
| 14 | { |

| | |
|---|---|
| 15 | RankedNormX[i] = normX[normRank[i]]; |
| 16 | RankedNormY[i] = normY[normRank[i]]; |

| | |
|---|---|
| 17 | } |
| 18 | lowess(RankedNormX, RankedNormY, normFeatNum, |

| | |
|---|---|
| 19 | f, nsteps, delta, |
| 20 | normFuncLR, normWeights, normResiduals); |

21 }

First, in the for-loop of lines 4-11, the array members “normX” and “normY” are initialized to contain the RTF comprising red-to-green log ratios for the logs of combined feature-signal magnitudes, as discussed above. Then, by the call to sort, on line 12, and the for-loop of lines 13-17, the RTF is converted into a monotonically increasing function stored in the array members “RankedNormX” and “RankedNormY.”

A LOWESS-based normalization is provided by the function member “LOWESS_norm:”

1 void normalizer::LOWESS_norm(double f)
2 {

| | |
|---|---|
| 3 | int i; |
| 4 | double x, C; |
| 5 | double newNormRed, newNormGreen; |
| 6 | for (i=0; i < red->getMaxNum( ); i++) |
| 7 | { |

| | |
|---|---|
| 8 | x = log10(red->getFeature(i) * green->getFeature(i))/2.0; |
| 9 | lowess_correct(RankedNormX, RankedNormY, |

| | |
|---|---|
| 10 | normFeatNum, x, f, C, normWeights); |

| | |
|---|---|
| 11 | newNormRed = red->getFeature(i) / pow(10, C / 2.0); |
| 12 | newNormGreen = green->getFeature(i) * pow(10, C / 2.0); |
| 13 | red->setFeature( newNormRed, i); |
| 14 | green->setFeature( newNormGreen, i); |

| | |
|---|---|
| 15 | } |

16 }

The function member “LOWESS_norm” normalizes the green and red data sets to one another by distributing the correction term computed by the LOWESS method between the green and red feature-signal magnitudes for each feature, as discussed above.

Finally, the function member “normalizer” has been rewritten to include calls to the LOWESS-based function members of the currently described embodiment:

1 void normalizer::normalize(bool arithmetic)
2 {

| | |
|---|---|
| 3 | if (OK) |
| 4 | { |

| | |
|---|---|
| 5 | if (arithmetic) arithmeticMean( ); |
| 6 | else geometricMean( ); |
| 7 | rankfilter(0.05); |
| 8 | LOWESS_smooth(0.25, 2, 0.0); |
| 9 | LOWESS_norm(0.25); |

| | |
|---|---|
| 10 | } |

11 }

Although the present invention has been described in terms of several particular embodiments, it is not intended that the invention be limited to these embodiments. Modifications within the spirit of the invention will be apparent to those skilled in the art. For example, an almost limitless number of different implementations of the method of the present invention are possible, including implementations that use different smoothing, curve-fitting, normalizing-function y-value estimation, and sorting methods. Different implementations may employ different modular organizations, data structures, control structures, and may be written in any number of different computer languages for execution on many different hardware/operating system platforms. The present invention may be applied to molecular array data preprocessed to flag outliers, subtract background, correct for known instrumental and experimental errors, and to otherwise prepare the data for subsequent analysis, although specific preprocessing is not necessary for practicing the present invention. Entire data sets may be normalized by the present invention, or portions of two or more data sets may be normalized by the methods of the present invention. Although the described embodiments focused on normalizing two data sets, the method of the present invention may be used to normalize more than two data sets. Normalization of more than two data sets can be accomplished in at least two different ways. First, the multiple data sets can be normalized in a pair-wise fashion, with overlap of members of the pairs ensuring that all data sets are normalized to a common standard. In one embodiment, the feature subset representing only features common to all data sets may be normalized, while in alternative embodiments, the union of features present in the multiple data sets may be normalized. Alternatively, the second described embodiment, in which computed differentials between estimated and measured feature-signal values are applied to both data sets, can be extended to be applicable to more than two data sets. In the extended embodiment, normalizing features may be selected based on hyper-dimensional rank-ordering neighborhoods, and the differentials between estimated and measured feature-signal magnitudes may be distributed amongst the multiple data sets in a way that maintains a constant combined feature-signal magnitude, as for the case of two data sets. Even considering a two-data-set case, there are a number of techniques for distributing computed differentials between estimated and measured feature-signal values between the two data sets, and, in multiple-data-set-cases, there are many different possible techniques for distributing computed differentials between estimated and measured feature-signal values between the multiple data sets. The method of the present invention may be used to normalize data sets in preparation for subsequent data-set analysis. Alternatively, the normalization method of the present invention may be used as a quality control step in monitoring performance of molecular array scanners, reproducibility of molecular arrays, reproducibility of molecular-array-based experiments. For example, if the normalization function before or after smoothing exhibits a percentage correction of feature signal-magnitudes in a feature signal-magnitude data set, which percentage varies over a range exceeding a predetermined value, or the normalization function otherwise varies for the feature signal-magnitudes in a feature signal-magnitude data set in some manner exceeding a predetermined limit. In such situations, an experimental result from which the feature signal-magnitude data sets were obtained, may be rejected, and the experiment (that is, the procedure or any part of it by which the feature signal-magnitude data sets were obtained from the array(s)) may be repeated (for example, the same array may be re-scanned, or another array with the same features may be exposed to a same sample then scanned).

There are alternative ways to perform rank ordering. For example, the feature signal magnitudes could be assigned ranks corresponding to percentile bins and normalizing features selected according to proximity of bin-based rank in the respective data sets.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously, many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A method for normalizing two data sets representing feature-signal magnitudes of a set of features of a molecular array, each data set comprising feature-signal-magnitudes associated with feature identifiers, the method comprising:
determining a rank of each feature with respect to an order of feature-signal magnitudes of a first feature-signal-magnitude data set;
determining a rank of each feature with respect to an order of feature-signal magnitudes of a second feature-signal-magnitude data set;
selecting, as a set of normalizing features, features from the set of features for which the ranks of the corresponding feature-signal-magnitudes within the feature-signal-magnitudes data sets are similar according to a similarity metric;
constructing a normalization function based on the feature-signal-magnitudes of the set of normalizing features; and
normalizing the two data sets using the normalization function.

2. The method of claim 1 wherein the first data set is obtained from the molecular array by optically scanning the features of the molecular array at a first wavelength, and the second data set is obtained from the molecular array by optically scanning the features of the molecular array at a second wavelength.

3. The method of claim 1 wherein there are at least one hundred differently ranked members in each of the feature data sets.

4. The method of claim 1 wherein the first data set is obtained from a first molecular array, and the second data set is obtained from a second molecular array with features corresponding to the features of the first molecular array.

5. The method of claim 1 applied to normalizing more than two data sets, wherein pairs of the more than two data sets are normalized by the method of claim 1.

6. The method of claim 5 further including:
normalizing a subset of features common to the data sets by the method of claim 1 to produce a calibrating set of features; and
normalizing each data set using a normalizing function derived from the calibrating feature set.

7. The method of claim 1 further including, prior to determining the rank of features in the first and second feature-signal-magnitude data sets by feature-signal-magnitude:
marking outlier feature-signal magnitudes in both data sets;
computing a distribution metric for each data set; and
globally normalizing each data set based on the computed distribution metric.

8. The method of claim 1 further including, prior to determining the rank of features in the first and second feature-signal-magnitude data sets by feature-signal magnitude:
identifying outlier-feature-signal-magnitudes in each set; wherein the rank of features is determined without considering the identified outlier features.

9. The method of claim 1 wherein only features not associated with corresponding outlier feature-signal magnitudes are selected for the set of normalizing features.

10. The method of claim 1 wherein selecting a set of normalizing features further includes:
removing from consideration as normalizing features those features for which one or both feature-signal magnitudes are outliers;
for each feature-signal magnitude position in the first ordered set of feature-signal-magnitudes representing the relative ranking of the feature-signal magnitude of a particular feature within the first data set,
determining whether a feature-signal magnitude corresponding to the particular feature is located at a second position within the second ordered set of feature-signal-magnitudes such that an absolute value difference between the feature-signal magnitude position in the first ordered set of feature-signal-magnitudes and the second position is less than a threshold value.

11. The method of claim 1 wherein the similarity metric is a distance between relative positions of the feature-signal magnitudes corresponding to a feature within the first and second ordered sets of feature-signal-magnitudes.

12. The method of claim 1 wherein constructing a normalization function based on the feature-signal-magnitudes of the set of normalizing features further includes:
constructing a numerical function that relates a ratio of feature-signal magnitudes corresponding to a normalizing feature within the first and second data sets to the feature-signal magnitude of the normalizing feature within the second data set.

13. The method of claim 12 further including smoothing the numerical function.

14. The method of claim 13 wherein normalizing the two data sets using the normalization function further includes:
for each feature,
setting a corresponding feature-signal magnitude in the second data set to the value of the numerical function corresponding to the feature-signal magnitude in the second data set multiplied by the feature-signal magnitude in the second data.

15. The method of claim 1 wherein constructing a normalization function based on the feature-signal-magnitudes of the set of normalizing features further includes:
constructing a numerical function that relates a ratio of feature-signal magnitudes corresponding to a normalizing feature within the first and second data sets to a combined feature-signal magnitude for the normalizing feature.

16. The method of claim 15 further including smoothing the numerical function using a modified LOWESS curve-fitting method.

17. The method of claim 16 wherein normalizing the two data sets using the normalization function further includes:
for each feature,
calculating a LOWESS-based combined feature-signal magnitude estimate using the numerical function;
calculating a differential between the LOWESS-based combined feature-signal magnitude estimate and a combined feature-signal magnitude for the feature; and
distributing the calculated differential between the feature-signal magnitudes in both data sets.

18. The method of claim 1 further including:
using one or more normalized data sets as a basis for evaluating operation of a molecular array scanner.

19. The method of claim 1 further including:
using one or more normalized data sets as a basis for evaluating the quality of a manufactured molecular array.

20. The method of claim 19 wherein a result is rejected based on the quality evaluation.

21. The method of claim 20 additionally comprising repeating an experiment based on the quality evaluation.

22. The method of claim 21 wherein the repeating includes re-scanning the array.

23. The method of claim 21 wherein the repeating includes exposing an array with the same features to a same sample.

24. The method of claim 1 further including:
using one or more normalized data sets as a basis for evaluating the reproducibility of a molecular-array-based experiment.

25. A representation of a data set, normalized by the method of claim 1 or derived from a data set normalized by the method of claim 1, printed in a human-readable format.

26. A computer program including an implementation of the method of claim 1.

27. A method comprising forwarding data representing a result obtained by the method of claim 1.

28. A method according to claim 27 wherein the data is communicated to a remote location.

29. A method comprising receiving data representing a result of a reading obtained by the method of claim 1.

30. A computer program product, comprising: a computer readable storage medium having a computer program stored thereon for performing the method of claim 1.

31. A method for normalizing more than two data sets, each representing feature-signal magnitudes of a set of features of one or more molecular arrays, each data set comprising feature-signal-magnitudes associated with feature identifiers, the method comprising:
for each data set, determining a rank of each feature with respect to an order of feature signal-magnitudes in the data set;
selecting, as a set of normalizing features, each feature from the set of features for which the ranks of the corresponding feature-signal-magnitudes within the sorted feature-signal-magnitude data sets fall within a neighborhood within a multi-dimensional rank space;
constructing a normalization function based on the feature-signal-magnitudes of the set of normalizing features; and
normalizing the more than two data sets using the normalization function.

32. The method of claim 31 wherein the more than two data sets are obtained from the one or more molecular arrays by optically scanning the features of the molecular array at different wavelengths.

33. The method of claim 31 wherein there are at least one hundred differently ranked members in each of the feature data sets.

34. The method of claim 31 further including, prior to determining the rank for each data feature-signal-magnitude data sets by feature-signal-magnitude:
marking outlier feature-signal magnitudes in the data sets;
computing a distribution metric for each data set; and
globally normalizing each data set based on the computed distribution metric.

35. The method of claim 31 wherein only features not associated with corresponding outlier feature-signal magnitudes are selected for the set of normalizing features.

36. The method of claim 31 wherein constructing a normalization function based on the feature-signal-magnitudes of the set of normalizing features further includes:
constructing a numerical function that relates the feature-signal magnitudes corresponding to a normalizing feature within the more than two data sets to a combined feature-signal magnitude for the normalizing feature.

37. The method of claim 36 further including smoothing the numerical function using a modified LOWESS curve-fitting method.

38. The method of claim 37 wherein normalizing the more than two data sets using the normalization function further includes:
for each feature,
calculating a LOWESS-based combined feature-signal magnitude estimate using the numerical function;
calculating a differential between the LOWESS-based combined feature-signal magnitude estimate and a combined feature-signal magnitude for the feature; and
distributing the calculated differential among the feature-signal magnitudes in the two or more data sets.

39. The method of claim 31 further including, prior to determining the rank of features in the first and second feature-signal-magnitude data sets by feature-signal magnitude:
identifying outlier-feature-signal-magnitudes in each set; wherein the rank of features is determined without considering the identified outlier features.

40. The method of claim 31 further including:
using one or more normalized data sets as a basis for evaluating operation of a molecular array scanner.

41. The method of claim 31 further including:
using one or more normalized data sets as a basis for evaluating the quality of a manufactured molecular array.

42. The method of claim 31 further including:
using one or more normalized data sets as a basis for evaluating the reproducibility of a molecular-array-based experiment.

43. The method of claim 41 wherein a result is rejected based on the quality evaluation.

44. A method comprising forwarding data representing a result obtained by the method of claim 31.

45. A method according to claim 44 wherein the data is communicated to a remote location.

46. A method comprising receiving data representing a result of a reading obtained by the method of claim 31.

47. A computer program product, comprising: a computer readable storage medium having a computer program stored thereon for performing the method of claim 31.

48. A system for processing molecular array data comprising:
a processor;
an input medium for receiving molecular array data; and
a program, executing on the processor, that performs a method of claim 31.
